(19) United States
(12) Patent Application Publication
(10) Pub. No.: US 2024/0327376 A1
(43) Pub. Date: Oct. 3, 2024

(54) PICOLINAMIDE COMPOUNDS AS SELECTIVE PHD1 INHIBITORS, COMPOSITIONS, AND METHODS OF USE

(71) Applicant: Akebia Therapeutics, Inc., Cambridge, MA (US)

(72) Inventors: Thomas P. BLAISDELL, Cambridge, MA (US); Paul E. FLEMING, Cambridge, MA (US)

(73) Assignee: Akebia Therapeutics, Inc., Cambridge, MA (US)

(21) Appl. No.: 18/742,035

(22) Filed: Jun. 13, 2024

Related U.S. Application Data
(63) Continuation of application No. PCT/IB2022/062394, filed on Dec. 16, 2022.
(60) Provisional application No. 63/291,048, filed on Dec. 17, 2021.

(51) Int. Cl. C07D 401/04; A61K 31/4439; C07D 213/81; C07D 405/14

(52) U.S. Cl. CPC C07D 401/04; A61K 31/4439; C07D 213/81; C07D 405/14

(57) ABSTRACT

The present invention provides, in part, novel small molecule inhibitors of PHD1 that are selective over PHD2 having a structure according to Formula (I), and sub-formulas thereof:

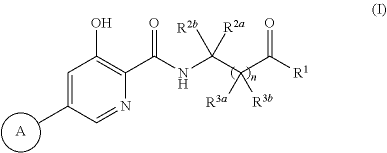

or a pharmaceutically acceptable salt thereof. The compounds provided herein can be useful for treatment of diseases including ischemia reperfusion injury (including but not limited to stroke, myocardial infarction, and acute kidney injury) inflammatory bowel disease, cancer (including colorectal cancer), and liver disease.

### CROSS-REFERENCE TO RELATED APPLICATIONS

The present application is a continuation of PCT Application No. PCT/IB2022/062394, filed Dec. 16, 2022, which claims the benefit of and priority to U.S. Provisional Application No. 63/291,048, filed Dec. 17, 2021, the contents of each of which are hereby incorporated by reference in their entireties.

### BACKGROUND

Hypoxia is a condition or state in which the supply of oxygen is insufficient for normal life function, for example, where there is low arterial oxygen supply. Hypoxia can lead to functional impairment of cells and structural tissue damage. The activation of cellular defense mechanisms during hypoxia is mediated by HIF (Hypoxia-inducible factor) protein. In response to hypoxic conditions, levels of HIFα are elevated in most cells because of a decrease in HIFα prolyl hydroxylation. Prolyl hydroxylation of HIFα is accomplished by a family of proteins variously termed the prolyl hydroxylase domain-containing proteins (PHD1, 2, and 3), also known as HIF prolyl hydroxylases (HPH-3, 2, and 1) or EGLN-2, 1, and 3. The PHD proteins are oxygen sensors and regulate the stability of HIF in an oxygen dependent manner. The three PHD isoforms function differently in their regulation of HIF and may have other non-HIF related regulatory roles.

Accordingly, compounds that can selectively inhibit one PHD isoform may be particularly beneficial in new, targeted therapies. For example, inhibition of PHD1 may be particularly beneficial for treating skeletal muscle cell degeneration (U.S. Pat. No. 7,858,593), for protection of myofibers against ischemia (Aragones et al. (2008) Nat. Genet. 40:170-80), for treatment of colitis and other forms of inflammatory bowel disease (Tambuwala et al. (2010) Gastroenterology 139:2093-101, and for treatment of heart failure and anemia in patients with concomitant cardiac and renal disease (Bao et al. (2010) J. Cardiovasc. Pharmacol. 56:147-55). Thus, there remains a need in the art for compounds that are selective inhibitors for PHD1.

### SUMMARY

The present invention provides, among other things, novel small molecule inhibitors of PHD1 that are selective over PHD2, PHD3, and other prolyl-4-hydroxylases and have utility for the treatment of diseases including ischemia reperfusion injury (including but not limited to stroke, myocardial infarction, and acute kidney injury) inflammatory bowel disease, cancer (including colorectal cancer), and liver disease.

In an aspect, provided herein are compounds having a structure according to Formula (I):

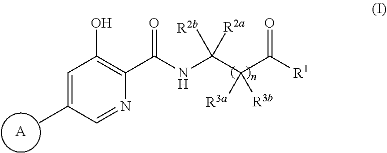

or a pharmaceutically acceptable salt thereof, wherein:
A is aryl or heteroaryl, optionally substituted with aryl, heteroaryl, halo, C₁-C₄ alkyl, alkoxy, arylalkoxy, heteroarylalkoxy, amino, arylamino, heteroarylamino, amide, cyano, nitro, sulfonamide;
R¹ is OH or optionally substituted ester;
R^{2a}, R^{2b}, R^{3a}, and R^{3b} are each independently H, OH, or C₁-C₄ alkyl, provided that at least one of R^{2a}, R^{2b} R^{3a}, or R^{3b} is OH;
or
R^{2a} and R^{2b} are independently H, OH, or C₁-C₄ alkyl; or R^{2a} and R^{2b} together with the carbon to which they are attached form a 3-6 membered cycloalkyl, which is optionally substituted with C₁-C₃ alkyl or substituted C₁-C₄ alkyl; or R^{2a} and R^{2b} together with the carbon to which they are attached form a 3-6 membered heterocycloalkyl, which is optionally substituted with C₁-C₃ alkyl; and R^{3a} and R^{3b} are independently H, OH, or C₁-C₄ alkyl; or R^{3a} and R^{3b} together with the carbon to which they are attached form a 3-6 membered cycloalkyl, which is optionally substituted with C₁-C₃ alkyl or substituted C₁-C₄ alkyl; or R^{3a} and R^{3b} together with the carbon to which they are attached form a 3-6 membered heterocycloalkyl, which is optionally substituted with C₁-C₃ alkyl;
n is 1 or 2; and
wherein at least one of R^{2a}, R^{2b}, R^{3a} and R^{3b} is not H.

In embodiments, R^{2a}, R^{2b}, R^{3a}, and R^{3b} are each independently H, OH, or C₁-C₄ alkyl, provided that at least one of R^{2a}, R^{2b} R^{3a}, or R^{3b} is OH.

In embodiments, R^{2a} and R^{2b} are independently H, OH, or C₁-C₄ alkyl; or R^{2a} and R^{2b} together with the carbon to which they are attached form a 3-6 membered cycloalkyl, which is optionally substituted with C₁-C₃ alkyl or substituted C₁-C₄ alkyl; or R^{2a} and R^{2b} together with the carbon to which they are attached form a 3-6 membered heterocycloalkyl, which is optionally substituted with C₁-C₃ alkyl; and R^{3a} and R^{3b} are independently H, OH, or C₁-C₄ alkyl; or R^{3a} and R^{3b} together with the carbon to which they are attached form a 3-6 membered cycloalkyl, which is optionally substituted with C₁-C₃ alkyl or substituted C₁-C₄ alkyl; or R^{3a} and R^{3b} together with the carbon to which they are attached form a 3-6 membered heterocycloalkyl, which is optionally substituted with C₁-C₃ alkyl.

In another aspect, provided herein are compounds having a structure according to Formula (II):

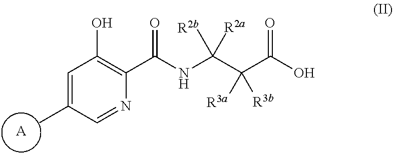

or a pharmaceutically acceptable salt thereof, wherein:
A is aryl or heteroaryl, optionally substituted with aryl, halo, C₁-C₄ alkyl, alkoxy, arylalkoxy, heteroarylalkoxy, amino, arylamino, heteroarylamino, amide, cyano, nitro, sulfonamide;
R^{2a}, R^{2b} R^{3a}, and R^{3b} are each independently H, OH, or C₁-C₄ alkyl, provided that at least one of R^{2a}, R^{2b} R^{3a}, or R^{3b} is OH; or
R^{2a} and R^{2b} are independently H, OH, or C₁-C₄ alkyl; or R^{2a} and R^{2b} together with the carbon to which they are attached form a 3-6 membered cycloalkyl, optionally substituted by substituted C₁-C₄ alkyl; or R^{2a} and R^{2b} together with the carbon to which they are attached form a 3-6 membered heterocycloalkyl; and R^{3a} and R^{3b} are independently H, OH, or C₁-C₄ alkyl; or R^{3a} and R^{3b} together with the carbon to which they are attached form a 3-6 membered cycloalkyl, optionally substituted by substituted C₁-C₄ alkyl; or R^{3a} and R^{3b} together with the carbon to which they are attached form a 3-6 membered heterocycloalkyl; and
wherein at least one of R^{2a}, R^{2b}, R^{3a} and R^{3b} is not H.

In some embodiments, A is

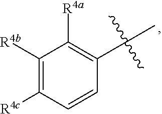

wherein
R^{4a}, R^{4b}, and R^{4c} are independently H, halo, aryl, heteroaryl, CH₂OR¹², OR¹², NHR¹² or CH₂R¹³;
R¹² is H, aryl optionally substituted with R¹⁴, or C₁-C₂ alkyl optionally substituted with R¹⁵.
R¹³ is heterocycloalkyl;
R¹⁴ is H or halo; and
R¹⁵ is cycloalkyl or aryl optionally substituted with halo.

In some embodiments, R¹³ is pyrrolidine.

In some embodiments, A is

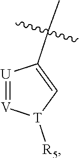

wherein
U, V, and T are independently CH, or N;
R⁵ is C₁-C₄ alkyl optionally substituted with R¹⁶; aryl optionally substituted with H, halo, CF₃; heteroaryl optionally substituted with CO₂R¹⁷; or heterocycloalkyl optionally substituted with CO₂R¹⁷;
R¹⁶ is H, aryl optionally substituted with halo, heterocycloalkyl; and
R¹⁷ is t-butyl.

In some embodiments, A is

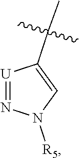

wherein
U is CH or N;
R⁵ is C₁-C₄ alkyl optionally substituted with R¹⁶; aryl optionally substituted with H, halo, or CF₃; heterocycloalkyl optionally substituted with CO₂R¹⁷; or heteroaryl optionally substituted with CO₂R¹⁷;
R¹⁶ is H, aryl optionally substituted with halo, or heterocycloalkyl; and
R¹⁷ is t-butyl.

In some embodiments, A is

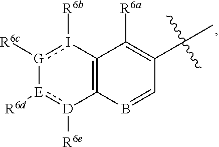

wherein
B, D, E, G, and I are independently C, CH, or N;
R^{6a}, R^{6b}, R^{6c}, and R^{6d} are independently H, C₁-C₃ alkyl, halo, OR¹⁸, or NHR¹⁹,
R^{6e} is H or ═O;
R¹⁸ is H, aryl optionally substituted with halo, or C₁-C₃ alkyl optionally substituted with R²⁰;
R¹⁹ is SO₂CH₃;
R²⁰ is aryl optionally substituted with halo; and
------ is an optional bond.

In some embodiments, A is

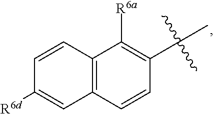

wherein
R^{6a} is H or methyl;
R^{6d} is H, OR¹⁸, or NHR¹⁹,
R¹⁸ is H, aryl optionally substituted with halo, or C₁-C₃ alkyl optionally substituted with R²⁰;
R¹⁹ is SO₂CH₃; and
R²⁰ is aryl optionally substituted with halo.

In some embodiments, A is

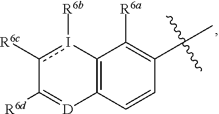

wherein
D is CH, CR^{6e} or N;
I is C, CH, or N;
R^{6a} is H or halo;
R^{6b} is H or C₁-C₃ alkyl;
R^{6c} is H, ═O or C₁-C₃ alkyl;
R^{6d} is H or C₁-C₃ alkyl;
R^{6e} is H or ═O; and
----- is an optional bond.

In some embodiments, A is

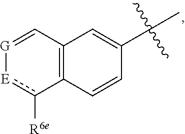

wherein
G is CH, or N;
E is CH, CH₂, N, or NH;
R^{6e} is H or ═O; and
----- is an optional bond.

In some embodiments, A is

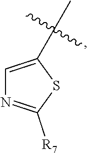

wherein
R⁷ is H, C₁-C₃ alkyl, or phenyl.

In some embodiments, A is

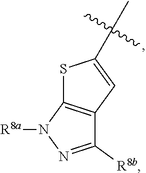

wherein
R^{8a} and R^{8b} are independently H or C₁-C₃ alkyl.

In some embodiments, A is

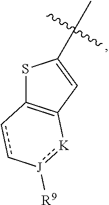

wherein
J is C, CH, or N;
K is CH, CH₂, N, or NH;
R⁹ is H, halo, C₁-C₄ alkyl, or CO₂R²¹;
R²¹ is t-butyl; and
------ is an optional bond.

In some embodiments, A is

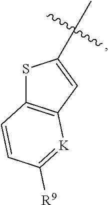

wherein
K is CH or N; and
R⁹ is H, halo, or C₁-C₄ alkyl.

In some embodiments, A is

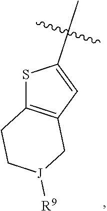

wherein
J is CH or N;
R⁹ is H or CO₂R²¹; and
R²¹ is t-butyl.

In some embodiments, A is

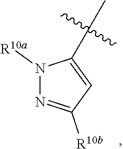

wherein
R^{10a} is H or C₁-C₃ alkyl; and
R^{10b} is H or thiazole.

In some embodiments, A is

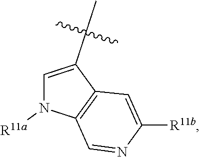

wherein
R^{11a} and R^{11b} are independently H, C₁-C₃ alkyl, or C₁-C₃ alkoxy.

In some embodiments, A is

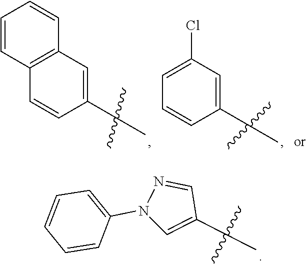 [SMILES: *c1ccc2ccccc2c1 ; *c1cccc(Cl)c1 ; *c1cnn(-c2ccccc2)c1]

In some embodiments, the compound is any one of Compounds 1-17, or a pharmaceutically acceptable salt thereof.

| Cmpd. | |
|---|---|
| No. | Structure |
| 1 | 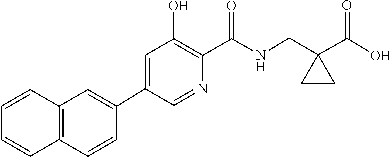 [SMILES: O=C(NCC1(C(=O)O)CC1)c1ncc(-c2ccc3ccccc3c2)cc1O] |
| 2 | 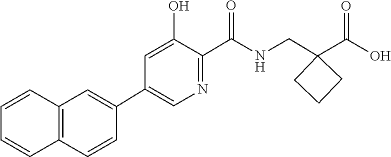 [SMILES: O=C(NCC1(C(=O)O)CCC1)c1ncc(-c2ccc3ccccc3c2)cc1O] |
| 3 | 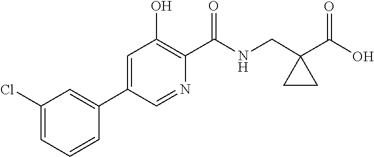 [SMILES: O=C(NCC1(C(=O)O)CC1)c1ncc(-c2cccc(Cl)c2)cc1O] |
| 4 | 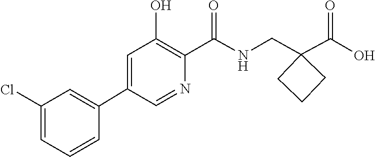 [SMILES: O=C(NCC1(C(=O)O)CCC1)c1ncc(-c2cccc(Cl)c2)cc1O] |
| 5 | 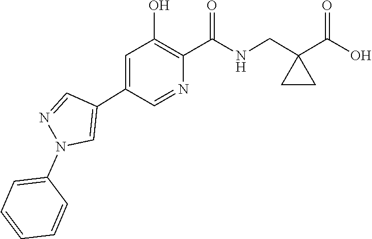 [SMILES: O=C(NCC1(C(=O)O)CC1)c1ncc(-c2cnn(-c3ccccc3)c2)cc1O] |
| 6 | 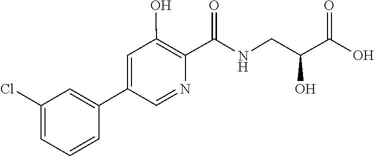 [SMILES: O=C(NC[C@H](O)C(=O)O)c1ncc(-c2cccc(Cl)c2)cc1O] |
| 7 | 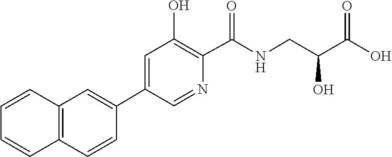 [SMILES: O=C(NC[C@H](O)C(=O)O)c1ncc(-c2ccc3ccccc3c2)cc1O] |
| 8 | 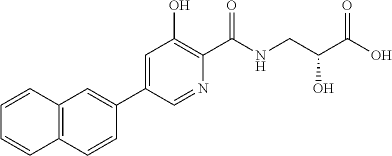 [SMILES: O=C(NC[C@@H](O)C(=O)O)c1ncc(-c2ccc3ccccc3c2)cc1O] |
| 9 | 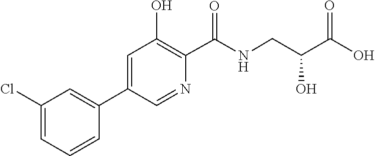 [SMILES: O=C(NC[C@@H](O)C(=O)O)c1ncc(-c2cccc(Cl)c2)cc1O] |
| 10 | 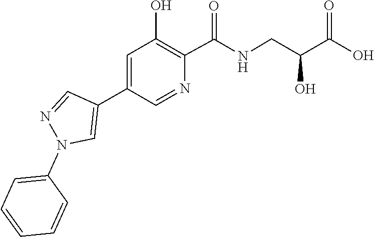 |
| 11 | 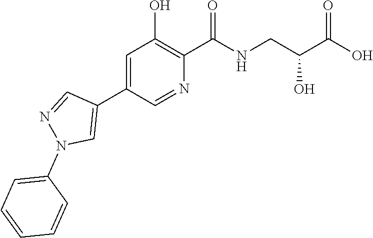 |
| 12 | 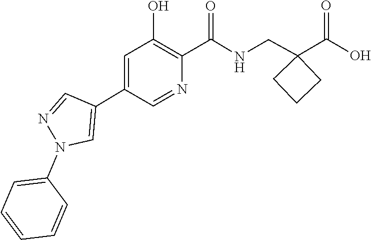 |
| 13 | 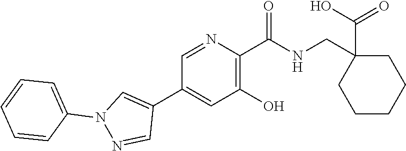 [SMILES: O=C(NCC1(C(=O)O)CCCCC1)c1ncc(-c2cnn(-c3ccccc3)c2)cc1O] |
| 14 | 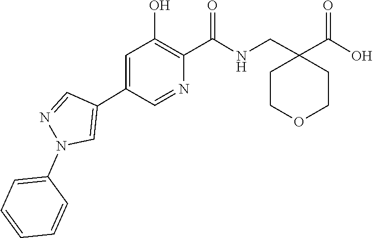 [SMILES: O=C(NCC1(C(=O)O)CCOCC1)c1ncc(-c2cnn(-c3ccccc3)c2)cc1O] |
| 15 | 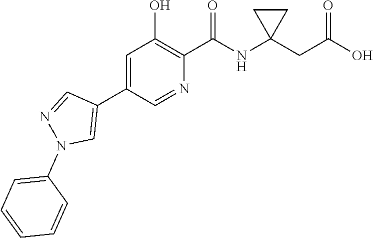 |
| 16 | 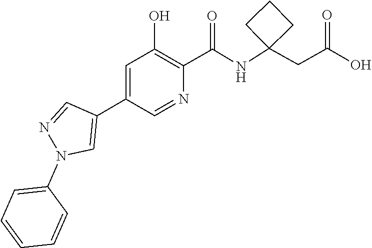 |
| 17 | 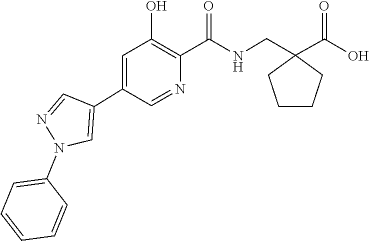 |

In some embodiments, in the compound of Formulas (I)-(IV) such as any one of Compounds 1-17 at least one hydrogen atom is replaced with a deuterium atom.

Also provided herein are methods for treating a disease mediated by PHD1 activity comprising administering to a subject a compound described herein (e.g., a compound of Formulas (I)-(IV) such as any one of Compounds 1-17). In some embodiments, disease mediated by PHD1 activity is ischemia reperfusion injury (e.g., stroke, myocardial infarction, acute kidney injury), IBD, cancer (e.g., colorectal cancer), liver disease, atherosclerosis, or cardiovascular disease.

### BRIEF DESCRIPTION OF DRAWINGS

FIG. 1 is an exemplary schematic illustration demonstrating the principle of the TR-FRET Assay for PHD enzymes (PHD1, PHD2, and PHD3). In the presence of 2-oxoglutarate and O₂, PHD enzyme hydroxylates proline 564 of biotin-tagged HIF-1α peptide resulting in generation of biotin-tagged HIF-1α-hydroxyproline, succinate and CO₂. The resulting proximity of the donor fluorophore complex, monoclonal antibody anti-6His-Terbium (Tb)-cryptate Gold, bound to the His-tagged VHL protein, EloB, EloC complex (His-VBC) and the acceptor fluorophore, SA-D2 complex, bound to HIF-1α-hydroxyproline results in a fluorescence resonance energy transfer signal that can be detected and quantified

### DETAILED DESCRIPTION OF THE DISCLOSURE

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, a bovine, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used in the description and the appended claims, the singular forms “a,” “an,” and “the” include plural referents unless the context clearly dictates otherwise. Thus, for example, reference to “a composition” includes mixtures of two or more such compositions.

Throughout the description and claims of this specification the word “comprise” and other forms of the word, such as “comprising” and “comprises,” means including but not limited to, and is not intended to exclude, for example, other additives, components, integers, or steps.

“Optional” or “optionally” means that the subsequently described event or circumstance may or may not occur, and that the description includes instances where the event or circumstance occurs and instances where it does not.

Improve, increase, or reduce: As used herein, the terms “improve,” “increase,” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio. Accordingly, pharmaceutically acceptable relates to substances that are not biologically or otherwise undesirable, i.e., the material can be administered to an individual along with the relevant active compound without causing clinically unacceptable biological effects or interacting in a deleterious manner with any of the other components of the pharmaceutical composition in which it is contained.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄-alkyl)₄ salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quarternization of an amine using an appropriate electrophile, e.g., an alkyl halide, to form a quarternized alkylated amino salt.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Whenever a term (e.g., alkyl or aryl) or either of their prefix roots (e.g., alk- or ar-) appear in a name of a substituent the name is to be interpreted as including those limitations provided herein. For example, affixing the suffix “-ene” to a group indicates the group is a divalent moiety, e.g., arylene is the divalent moiety of aryl, and heteroarylene is the divalent moiety of heteroaryl. Similarly, affixing the suffix “-oxy” to a group indicates the group is attached to the parent molecular structure through an oxygen atom (—O—).

Aliphatic: As used herein, the term aliphatic refers to C₁-C₄₀ hydrocarbons and includes both saturated and unsaturated hydrocarbons. An aliphatic may be linear, branched, or cyclic. For example, C₁-C₂₀ aliphatics can include C₁-C₂₀ alkyls (e.g., linear or branched C₁-C₂₀ saturated alkyls), C₂-C₂₀ alkenyls (e.g., linear or branched C₄-C₂₀ dienyls, linear, or branched C₆-C₂₀ trienyls, and the like), and C₂-C₂₀ alkynyls (e.g., linear or branched C₂-C₂₀ alkynyls). C₁-C₂₀ aliphatics can include C₃-C₂₀ cyclic aliphatics (e.g., C₃-C₂₀ cycloalkyls, C₄-C₂₀ cycloalkenyls, or C₈-C₂₀ cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the aliphatic is unsubstituted. In some embodiments, the aliphatic does not include any heteroatoms.

Alkyl: As used herein, the term “alkyl” means acyclic linear and branched hydrocarbon groups, e.g. “C₁-C₂₀ alkyl” refers to alkyl groups having 1-20 carbons and “C₁-C₄ alkyl” refers to alkyl groups having 1-4 carbons. Alkyl groups include C₁-C₂₀ alkyl, C₁-C₁₅ alkyl, C₁-C₁₀ alkyl, C₁-C₄ alkyl, and C₁-C₃ alkyl). In embodiments, an alkyl group is C₁-C₄ alkyl. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, C₁-C₄ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkyl is substituted (e.g., with 1, 2, 3, 4, 5, or 6 substituent groups as described herein). In some embodiments, an alkyl group is substituted with a-OH group and may also be referred to herein as a “hydroxyalkyl” group, where the prefix denotes the —OH group and “alkyl” is as described herein. In some embodiments, the alkyl is substituted with a —OR′ group.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms.

Alkenyl: As used herein, “alkenyl” means any linear or branched hydrocarbon chains having one or more unsaturated carbon-carbon double bonds that may occur in any stable point along the chain, e.g. “C₂-C_{2M} alkenyl” refers to an alkenyl group having 2-20 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In some embodiments, the alkenyl comprises 1, 2, or 3 carbon-carbon double bond. In some embodiments, the alkenyl comprises a single carbon-carbon double bond. In some embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkenyl is unsubstituted. In some embodiments, the alkenyl is substituted (e.g., with 1, 2, 3, 4, 5, or 6 substituent groups as described herein). In some embodiments, an alkenyl group is substituted with a-OH group and may also be referred to herein as a “hydroxyalkenyl” group, where the prefix denotes the —OH group and “alkenyl” is as described herein.

Alkynyl: As used herein, “alkynyl” means any hydrocarbon chain of either linear or branched configuration, having one or more carbon-carbon triple bonds occurring in any stable point along the chain, e.g. “C₂-C₂₀ alkynyl” refers to an alkynyl group having 2-20 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In some embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkynyl is unsubstituted. In some embodiments, the alkynyl is substituted (e.g., with 1, 2, 3, 4, 5, or 6 substituent groups as described herein).

Alkoxy: The term “alkoxy” refers to the group —O-alkyl, including from 1 to 10 carbon atoms of a straight, branched, saturated cyclic configuration and combinations thereof, attached to the parent molecular structure through an oxygen. Examples include methoxy, ethoxy, propoxy, isopropoxy, butoxy, t-butoxy, pentoxy, cyclopropyloxy, cyclohexyloxy and the like. “Lower alkoxy” refers to alkoxy groups containing one to six carbons. In some embodiments, C₁₋₄ alkoxy is an alkoxy group which encompasses both straight and branched chain alkyls of from 1 to 4 carbon atoms. Unless stated otherwise in the specification, an alkoxy group can be optionally substituted by one or more substituents (e.g., as described herein for alkyl). The terms “alkenoxy” and “alkynoxy” mirror the above description of “alkoxy” wherein the prefix “alk” is replaced with “alken” or “alkyn” respectively, and the parent “alkenyl” or “alkynyl” terms are as described herein.

Amino: The term “amino” or “amine” refers to a —N(R′)₂ group, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated otherwise in the specification, each of which moiety can itself be optionally substituted as described herein, or two R′ can combine with the nitrogen atom to form a 3-, 4-, 5-, 6-, or 7-membered ring. In embodiments, an amino group is —NHR′, where R′ is aryl (“arylamino”), heteroaryl (“heteroarylamino”), or alkyl (“alkylamino”).

Amide: The term “amide” or “amido” refers to a chemical moiety with formula —C(O)N(R′)₂, —C(O)N(R′)—, —NR′C(O)R′, or —NR′C(O)—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In some embodiments, an aryl group has 6 ring carbon atoms (“C₆ aryl,” e.g., phenyl). In some embodiments, an aryl group has 10 ring carbon atoms (“C₁₀ aryl,” e.g., naphthyl such as 1-naphthyl and 2-naphthyl). In some embodiments, an aryl group has 14 ring carbon atoms (“C₁₄ aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

Arylalkyl: The term “arylalkyl” refers to an -(alkylene)-aryl radical where aryl and alkylene are as disclosed herein and which are optionally substituted by one or more of the exemplary substituent groups described herein. The “arylalkyl” group is bonded to the parent molecular structure through the alkylene moiety. The term “arylalkoxy” refers to an —O-[arylalkyl] radical (—O-[(alkylene)-aryl]), which is attached to the parent molecular structure through the oxygen.

Arylene: The term “arylene” as used herein refers to an aryl group that is divalent (that is, having two points of attachment to the molecule). Exemplary arylenes include phenylene (e.g., unsubstituted phenylene or substituted phenylene).

Cyclic: The term “cyclic” as used herein, refers to any covalently closed structure. Cyclic moieties include, for example, carbocycles (e.g., aryls and cycloalkyls), heterocycles (e.g., heteroaryls and heterocycloalkyls), aromatics (e.g. aryls and heteroaryls), and non-aromatics (e.g., cycloalkyls and heterocycloalkyls). In some embodiments, cyclic moieties are optionally substituted. In some embodiments, cyclic moieties form part of a ring system.

Cycloaliphatic: The term “cycloaliphatic” refers to a monocyclic or polycyclic radical that contains only carbon and hydrogen, and can be saturated or partially unsaturated. Fully saturated cycloaliphatics can be termed “cycloalkyl”. Partially unsaturated cycloalkyl groups can be termed “cycloalkenyl” if the carbocycle contains at least one double bond, or “cycloalkynyl” if the carbocycle contains at least one triple bond. Cycloaliphatic groups include groups having from 3 to 13 ring atoms (e.g., C₃₋₁₃ cycloalkyl). Whenever it appears herein, a numerical range such as “3 to 10” refers to each integer in the given range; e.g., “3 to 10 carbon atoms” means that the cycloaliphatic group (e.g., cycloalkyl) can consist of 3 carbon atoms, 4 carbon atoms, 5 carbon atoms, etc., up to and including 10 carbon atoms. The term “cycloaliphatic” also includes bridged and spiro-fused cyclic structures containing no heteroatoms. The term also includes monocyclic or fused-ring polycyclic (i.e., rings which share adjacent pairs of ring atoms) groups. Polycyclic cycloaliphatic groups include bicycles, tricycles, tetracycles, and the like. In some embodiments, “cycloalkyl” can be a C₃₋₈ cycloalkyl group. In some embodiments, “cycloalkyl” can be a C₃₋₅ cycloalkyl group. Illustrative examples of cycloaliphatic groups include, but are not limited to the following moieties: C₃₋₆ cycloaliphatic groups include, without limitation, cyclopropyl (C₃), cyclobutyl (C₄), cyclopentyl (C₅), cyclopentenyl (C₅), cyclohexyl (C₆), cyclohexenyl (C), cyclohexadienyl (C) and the like. Examples of C₃₋₇ cycloaliphatic groups include norbornyl (C₇). Examples of C₃₋₈ cycloaliphatic groups include the aforementioned C₃₋₇ carbocyclyl groups as well as cycloheptyl(C₇), cycloheptadienyl (C₇), cyclohept-atrienyl (C₇), cyclooctyl (C₈), bicyclo[2.2.1]heptanyl, bicyclo[2.2.2]octanyl, and the like. Examples of C₃₋₁₃ cycloaliphatic groups include the aforementioned C₃₋₈ carbocyclyl groups as well as octahydro-1H-indenyl, decahydronaphthalenyl, spiro[4.5]decanyl, and the like.

Cyano: The term “cyano” refers to a —CN group.

Deuterium: The term “deuterium” is also called heavy hydrogen. Deuterium is isotope of hydrogen with a nucleus consisting of one proton and one neutron, which is double the mass of the nucleus of ordinary hydrogen (one proton). In embodiments, deuterium can also be identified as ²H.

Ester: The term “ester” refers to a group of formula —C(O)OR′ or —R′OC(O)—, where R′ is selected from alkyl, alkenyl, alkynyl, heteroalkyl (bonded through a chain carbon), cycloalkyl, aryl, arylalkyl, heteroaryl, heteroarylalkyl, or heterocycloalkyl as described herein.

Halogen or Halo: As used herein, the term “halogen” or “halo” means fluorine, chlorine, bromine, or iodine.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl. Accordingly, the term “heteroalkoxy” refers to the group —O-heteroalkyl, where the group is attached to the parent molecular structure via the oxygen.

Heteroalkylene: The term “heteroalkylene,” as used herein, represents a divalent form of a heteroalkyl group as described herein.

Heteroaryl: The term “heteroaryl,” as used herein, refers to a monocyclic, bicyclic, or tricyclic carbocyclic ring system having a total of six to fourteen ring members, wherein said ring system has a single point of attachment to the rest of the molecule, wherein at least one ring in the system is aromatic, wherein each ring in the system contains 4 to 7 ring members, and wherein at least one ring atom is a heteroatom such as, but not limited to, nitrogen and oxygen. Examples of heteroaryl groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Accordingly, the term “heteroaryloxy” refers to the group —O-heteroaryl, where the group is attached to the parent molecular structure via the oxygen.

Heteroarylalkyl: The term “heteroarylalkyl” refers to an -(alkylene)-heteroaryl radical where heteroaryl and alkylene are as disclosed herein and which are optionally substituted by one or more of the exemplary substituent groups described herein. The “heteroarylalkyl” group is bonded to the parent molecular structure through the alkylene moiety. The term “heteroarylalkoxy” refers to an —O-[heteroarylalkyl] radical (—O-[(alkylene)-heteroaryl]), which is attached to the parent molecular structure through the oxygen.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. Examples of heterocycloalkyl groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, and quinolizinyl. The heterocycloalkyl group can be substituted or unsubstituted.

Heterocycle: The term “heterocycle” refers to heteroaryl and heterocycloalkyl as used herein, refers to groups containing one to four heteroatoms each selected from O, S, and N, wherein each heterocycle group has from 4 to 10 atoms in its ring system, and with the proviso that the ring of said group does not contain two adjacent O or S atoms. Herein, whenever the number of carbon atoms in a heterocycle is indicated (e.g., C₁-C₆-heterocycle), at least one other atom (the heteroatom) must be present in the ring. Designations such as “C₁-C₆-heterocycle” refer only to the number of carbon atoms in the ring and do not refer to the total number of atoms in the ring. In some embodiments, it is understood that the heterocycle ring has additional heteroatoms in the ring. Designations such as “4-6-membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In some embodiments, in heterocycles that have two or more heteroatoms, those two or more heteroatoms are the same or different from one another. In some embodiments, heterocycles are optionally substituted. In some embodiments, binding to a heterocycle is at a heteroatom or via a carbon atom. Heterocycloalkyl groups include groups having only 4 atoms in their ring system, but heteroaryl groups must have at least 5 atoms in their ring system. The heterocycle groups include benzo-fused ring systems. An example of a 4-membered heterocycle group is azetidinyl (derived from azetidine). An example of a 5-membered heterocycle group is thiazolyl. An example of a 6-membered heterocycle group is pyridyl, and an example of a 10-membered heterocycle group is quinolinyl. In some embodiments, the foregoing groups, as derived from the groups listed above, are C-attached or N-attached where such is possible. For instance, in some embodiments, a group derived from pyrrole is pyrrol-1-yl (N-attached) or pyrrol-3-yl (C-attached). Further, in some embodiments, a group derived from imidazole is imidazol-1-yl or imidazol-3-yl (both N-attached) or imidazol-2-yl, imidazol-4-yl or imidazol-5-yl (all C-attached). The heterocycle groups include benzo-fused ring systems and ring systems substituted with one or two oxo (═O) moieties such as pyrrolidin-2-one. In some embodiments, depending on the structure, a heterocycle group is a monoradical or a diradical (i.e., a heterocyclene group). The heterocycles described herein are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alynyl, carboxy, cyano, formyl, haloalkoxy, haloalkyl, halogen, hydroxyl, hydroxyalkylene, mercapto, nitro, amino, and amido moities.

Isotope: The term “isotope” refers to a variant of a particular chemical element which differs in neutron number, and consequently in nucleon number. All isotopes of a given element have the same number of protons but different numbers of neutrons in each atom.

Nitro: The term “nitro” refers to a —NO₂ group.

Sulfonamide: The term “sulfonamide” or sulfonamido” refers to the following groups: —S(═O)₂—(R′)₂, —N(R′)—S(═O)₂—R′, —S(═O)₂—N(R′)—, or —N(R′)—S(═O)₂—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Moiety: The term “moiety” refers to a specific segment or functional group of a molecule. Chemical moieties are often recognized chemical entities embedded in or appended to a molecule.

Molecular groups herein may be substituted or unsubstituted (e.g., as described herein). The term “substituted” means that the specified group or moiety bears one or more substituents: at least one hydrogen present on a group atom (e.g., a carbon or nitrogen atom) is replaced with a permissible substituent, e.g., a substituent which upon substitution for the hydrogen results in a stable compound, e.g., a compound which does not spontaneously undergo transformation such as by rearrangement, cyclization, elimination, or other reaction. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In embodiments, a group described herein is substituted. In embodiments, a group described herein is unsubstituted. In cases where a specified moiety or group is not expressly noted as being optionally substituted or substituted with any specified substituent, it is understood that such a moiety or group is intended to be unsubstituted.

A wide variety of substituents are well known, and methods for their formation and introduction into a variety of parent groups are also well known. Representative substituents include but are not limited to alkyl, cycloalkyl, alkenyl, cycloalkenyl, alkynyl, arylalkyl, alkylaryl, aryl, arylalkoxy, arylamino, heteroarylamino, heteroaryl, heteroarylalkoxy, heterocycloalkyl, hydroxyalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, amide, cyano, alkoxy, hydroxy, sulfonamide, halo (e.g., Cl and Br), nitro, oximino, COOR⁵⁰, COR⁵⁰, SO₀₋₂R⁵⁰, SO₂NR⁵⁰R⁵¹, NR⁵²SO₂R⁵⁰, ═C(R⁵⁰R⁵¹), ═N—OR⁵⁰, ═N—CN, ═C(halo)₂, ═S, ═O, CON(R⁵⁰R⁵¹), OCOR⁵⁰, —OCON(R⁵⁰R⁵¹), N(R⁵²)CO(R⁵⁰), N(R⁵²)COOR⁵⁰ and N(R⁵²)CON(R⁵⁰(R⁵¹), wherein R⁵⁰, R⁵¹ and R⁵² may be independently selected from the following: a hydrogen atom and a branched or straight-chain, C₁₋₆-alkyl, C₃₋₆-cycloalkyl, C₄₋₆-heterocycloalkyl, heteroaryl and aryl group, with or without substituents. When permissible, R⁵⁰ and R⁵¹ can be joined together to form a carbocyclic or heterocyclic ring system.

In preferred embodiments, the substituent is selected from halogen, —COR′, —CO₂H, —CO₂R′, —CN, —OH, —OR′, —OCOR′, —OCO₂R′, —NH₂, —NHR′, —N(R′)₂, —SR′, and —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In certain embodiments thereof, R′ independently is an unsubstituted alkyl (e.g., unsubstituted C₁-C₂₀ alkyl, C₁-C₁₅ alkyl, C₁-C₁₀ alkyl, or C₁-C₃ alkyl). Preferably, R′ independently is unsubstituted C₁-C₃ alkyl.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

### Compounds of the Invention

Disclosed herein are compounds that are potent inhibitors of PHD1. In some embodiments, the compounds of the present invention have enzymatic half maximal inhibitory concentration (IC₅₀) values of less than 100 μM against PHD1. In some embodiments, the compounds of the present invention have an IC₅₀ value of less than 50 μM against PHD1. In some embodiments, the compounds of the present invention have an IC₅₀ value of less than 25 μM against PHD1. In some embodiments, the compounds of the present invention have an IC₅₀ value of less than 20 μM against PHD1. In some embodiments, the compounds of the present invention have an IC₅₀ value of less than 15 μM against PHD1. In some embodiments, the compounds of the present invention have an IC₅₀ value of less than 10 μM against PHD1. In some embodiments, the compounds of the present invention have an IC₅₀ value of less than 5 μM against PHD1. In some embodiments, the compounds of the present invention have an IC₅₀ value of less than 1 μM against PHD1. In some embodiments, the compounds of the present invention have an IC₅₀ value of about 3 nM to about 5 nM against PHD1. In some embodiments, the compounds of the present invention have an IC₅₀ value of about 5 nM to about 10 nM against PHD1. In some embodiments, the compounds of the present invention have an IC₅₀ value of about 10 nM to about 20 nM against PHD1. In some embodiments, the compounds of the present invention have an IC₅₀ value of about 20 nM to about 50 nM against PHD1. In some embodiments, the compounds of the present invention have an IC₅₀ value of about 50 nM to about 100 nM against PHD1. In some embodiments, the compounds of the present invention have an IC₅₀ value of about 100 nM to about 200 nM against PHD1. In some embodiments, the compounds of the present invention have an IC₅₀ value of about 200 nM to about 500 nM against PHD1. In some embodiments, the compounds of the present invention have an IC₅₀ value of about 500 nM to about 1000 nM against PHD1.

Disclosed herein are a series of inhibitors that are potent inhibitors of PHD1, which unexpectedly show an increase in selectivity for PHD1 over PHD2. In some embodiments, the selectivity for PHD1 over PHD2 is about 2 to about 1500 fold. In some embodiments, the selectivity for PHD1 over PHD2 is about 2 to about 10 fold, about 10 to about 20 fold, about 20 to about 50 fold, about 50 to about 100 fold, about 100 to about 200 fold, about 200 to about 500 fold, about 500 to about 1000 fold, about 1000 to about 1500 fold. In some embodiments, the selectivity for PHD1 over PHD2 is about or greater than 2 fold, about or greater than 5 fold, about or greater than 10 fold, about or greater than 20 fold, about or greater than 30 fold, about or greater than 40 fold, about or greater than 50 fold, about or greater than 75 fold, about or greater than 100 fold, about or greater than 150 fold, about or greater than 200 fold, about or greater than 500 fold, and about or greater than 1000 fold.

Representative examples from this class show inhibitory activity and selectivity for PHD1 in vitro.

Exemplary compounds are described herein. In particular, these selective inhibitors can feature a substituted alkylene moiety (e.g., an alkylene substituted with a hydroxy or a cyclic group) linking the amide NH with a carboxyl moiety.

### Compounds of Formulas (I)-(IV)

In an aspect, provided herein are compounds having a structure according to Formula (I):

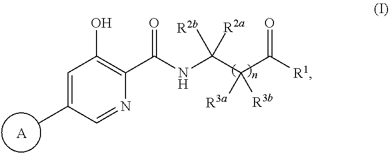

or a pharmaceutically acceptable salt thereof, wherein:
A is aryl or heteroaryl, optionally substituted with aryl, heteroaryl, halo, C₁-C₄ alkyl, alkoxy, aryloxy, heteroaryloxy, amino, arylamino, heteroarylamino, amide, cyano, nitro, sulfonamide;
R¹ is OH or optionally substituted ester;
R^{2a}, R^{2b} R^{3a}, and R^{3b} are each independently H, OH, C₁-C₄ alkyl, provided that at least one of R^{2a}, R^{2b} R^{3a}, or R^{3b} is OH; or
R^{2a} and R^{2b} are independently H, OH, or C₁-C₄ alkyl; or R^{2a} and R^{2b} together with the carbon to which they are attached form a 3-6 membered cycloalkyl, which is optionally substituted with C₁-C₃ alkyl or substituted C₁-C₄ alkyl; or R^{2a} and R^{2b} together with the carbon to which they are attached form a 3-6 membered heterocycloalkyl, which is optionally substituted with C₁-C₃ alkyl; and R^{3a} and R^{3b} are independently H, OH, or C₁-C₄ alkyl; or R^{3a} and R^{3b} together with the carbon to which they are attached form a 3-6 membered cycloalkyl, which is optionally substituted with C₁-C₃ alkyl or substituted C₁-C₄ alkyl; or R^{3a} and R^{3b} together with the carbon to which they are attached from a 3-6 membered heterocycloalkyl, which is optionally substituted with C₁-C₃ alkyl;
n is 1 or 2; and
wherein at least one of R^{2a}, R^{2b}, R^{3a} and R^{3b} is not H.

In embodiments, R^{2a}, R^{2b} R^{3a}, and R^{3b} are each independently H, OH, C₁-C₄ alkyl, provided that at least one of R^{2a}, R^{2b} R^{3a}, or R^{3b} is OH.

In embodiments, R^{2a} and R^{2b} are independently H, OH, or C₁-C₄ alkyl; or R^{2a} and R^{2b} together with the carbon to which they are attached form a 3-6 membered cycloalkyl, which is optionally substituted with C₁-C₃ alkyl or substituted C₁-C₄ alkyl; or R^{2a} and R^{2b} together with the carbon to which they are attached form a 3-6 membered heterocycloalkyl, which is optionally substituted with C₁-C₃ alkyl; and R^{3a} and R^{3b} are independently H, OH, or C₁-C₄ alkyl; or R^{3a} and R^{3b} together with the carbon to which they are attached form a 3-6 membered cycloalkyl, which is optionally substituted with C₁-C₃ alkyl or substituted C₁-C₄ alkyl; or R^{3a} and R^{3b} together with the carbon to which they are attached from a 3-6 membered heterocycloalkyl, which is optionally substituted with C₁-C₃ alkyl.

In embodiments, R^{2a} and R^{2b} are independently H, OH, or C₁-C₄ alkyl.

In embodiments, R^{2a} and R^{2b} together with the carbon to which they are attached form a 3-6 membered cycloalkyl, which is optionally substituted with C₁-C₃ alkyl or substituted C₁-C₄ alkyl.

In embodiments, R^{2a} and R^{2b} together with the carbon to which they are attached form a 3-6 membered heterocycloalkyl, which is optionally substituted with C₁-C₃ alkyl.

In embodiments, R^{3a} and R^{3b} are independently H, OH, or C₁-C₄ alkyl.

In embodiments, R^{3a} and R^{3b} together with the carbon to which they are attached form a 3-6 membered cycloalkyl, which is optionally substituted with C₁-C₃ alkyl or substituted C₁-C₄ alkyl.

In embodiments, R^{3a} and R^{3b} together with the carbon to which they are attached from a 3-6 membered heterocycloalkyl, which is optionally substituted with C₁-C₃ alkyl.

In another aspect, provided herein are compounds having a structure according to Formula (II):

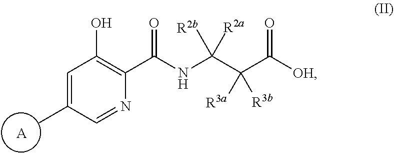

or a pharmaceutically acceptable salt thereof, wherein:
A is aryl or heteroaryl, optionally substituted with aryl, halo, C₁-C₄ alkyl, alkoxy, aryloxy, heteroaryloxy, amino, arylamino, heteroarylamino, amide, cyano, nitro, sulfonamide;
R^{2a}, R^{2b} R^{3a}, and R^{3b} are each independently H, OH, C₁-C₄ alkyl, provided that at least one of R^{2a}, R^{2b} R^{3a}, or R^{3b} is OH; or
R^{2a} and R^{2b} are independently H, OH, or C₁-C₄ alkyl; or R^{2a} and R^{2b} together with the carbon to which they are attached form a 3-6 membered cycloalkyl, optionally substituted by substituted C₁-C₄ alkyl; or R^{2a} and R^{2b} together with the carbon to which they are attached form a 3-6 membered heterocycloalkyl; and R^{3a} and R^{3b} are independently H, OH, or C₁-C₄ alkyl; or R^{3a} and R^{3b} together with the carbon to which they are attached form a 3-6 membered cycloalkyl, optionally substituted by substituted C₁-C₄ alkyl; or R^{3a} and R^{3b} together with the carbon to which they are attached form a 3-6 membered heterocycloalkyl; and
wherein at least one of R^{2a}, R^{2b}, R^{3a} and R^{3b} is not H.

In embodiments, R^{2a}, R^{2b} R^{3a}, and R^{3b} are each independently H, OH, C₁-C₄ alkyl, provided that at least one of R^{2a}, R^{2b} R^{3a}, or R^{3b} is OH.

In embodiments, R^{2a} and R^{2b} are independently H, OH, or C₁-C₄ alkyl; or R^{2a} and R^{2b} together with the carbon to which they are attached form a 3-6 membered cycloalkyl, optionally substituted by substituted C₁-C₄ alkyl; or R^{2a} and R^{2b} together with the carbon to which they are attached form a 3-6 membered heterocycloalkyl; and R^{3a} and R^{3b} are independently H, OH, or C₁-C₄ alkyl; or R^{3a} and R^{3b} together with the carbon to which they are attached form a 3-6 membered cycloalkyl, optionally substituted by substituted C₁-C₄ alkyl; or R^{3a} and R^{3b} together with the carbon to which they are attached form a 3-6 membered heterocycloalkyl.

In embodiments, R^{2a} and R^{2b} are independently H, OH, or C₁-C₄ alkyl.

In embodiments, R^{2a} and R^{2b} together with the carbon to which they are attached form a 3-6 membered cycloalkyl, optionally substituted by substituted C₁-C₄ alkyl.

In embodiments, R^{2a} and R^{2b} together with the carbon to which they are attached form a 3-6 membered heterocycloalkyl.

In embodiments, R^{3a} and R^{3b} are independently H, OH, or C₁-C₄ alkyl; or R^{3a} and R^{3b} together with the carbon to which they are attached form a 3-6 membered cycloalkyl, optionally substituted by substituted C₁-C₄ alkyl.

In embodiments, R^{3a} and R^{3b} together with the carbon to which they are attached form a 3-6 membered heterocycloalkyl.

In some embodiments, R^{3a} and R^{3b} together with the carbon to which they are attached form a 3-membered cycloalkyl.

In some embodiments, R^{3a} and R^{3b} together with the carbon to which they are attached form a 4-membered cycloalkyl.

In some embodiments, R^{3a} and R^{3b} together with the carbon to which they are attached form a 5-membered cycloalkyl.

In some embodiments, R^{3a} and R^{3b} together with the carbon to which they are attached form a 6-membered cycloalkyl.

In some embodiments, R^{3a} and R^{3b} together with the carbon to which they are attached form a 3-membered heterocycloalkyl.

In some embodiments, R^{3a} and R^{3b} together with the carbon to which they are attached form a 4-membered heterocycloalkyl.

In some embodiments, R^{3a} and R^{3b} together with the carbon to which they are attached form a 5-membered heterocycloalkyl.

In some embodiments, R^{3a} and R^{3b} together with the carbon to which they are attached form a 6-membered heterocycloalkyl.

In some embodiments, R^{2a} and R^{2b} are both H.

In some embodiments, one of R^{3a} and R^{3b} is H, and the other is OH, or R^{3a} and R^{3b} combine to form optionally substituted 3-6-membered cycloalkyl or 3-6-membered heterocycloalkyl.

In some embodiments, one of R^{3a} and R^{3b} is H, and the other is OH. In some embodiments, the carbon substituted by R^{3a} and R^{3b} has the S-configuration. In some embodiments, the carbon substituted by R^{3a} and R^{3b} has the R-configuration.

In some embodiments, a compound of Formula (I) or (II) has the following structure,

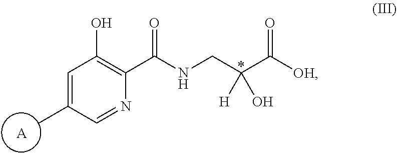

or a pharmaceutically acceptable salt thereof, wherein A is as defined anywhere herein.

In some embodiments, the carbon substituted by the asterisk has the S-configuration. In some embodiments, the carbon substituted by the asterisk has the R-configuration.

In some embodiments, R^{3a} and R^{3b} combine to form optionally substituted 3-6-membered cycloalkyl. In some embodiments, R^{3a} and R^{3b} combine to form an unsubstituted 3-6-membered cycloalkyl. In some embodiments, R^{3a} and R^{3b} combine to form cyclopropyl, cyclobutyl, cyclopenyl, or cyclohexyl.

In some embodiments, a compound of Formula (I) or (II) has the following structure,

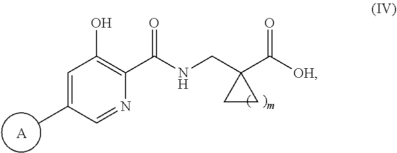

or a pharmaceutically acceptable salt thereof, wherein m is independently 1, 2, 3, or 4, and A is as defined anywhere herein. In some embodiments, m is 1. In some embodiments, m is 2. In some embodiments, m is 3. In some embodiments, m is 4.

In some embodiments, A is an optionally substituted phenyl.

In some embodiments, A is an optionally substituted naphthyl.

In some embodiments, A is an optionally substituted 5-membered heteroaryl.

In some embodiments, A is an optionally substituted bicyclic heteroaryl (e.g., an 7- to 9-membered heteroaryl).

In some embodiments, A is an optionally substituted group selected from: phenyl, pyrrolyl, imidazolyl, triazolyl, naphthyl, quinolyl, isoquinolyl, quinoxalyl, phthalazinyl, thiazolyl, thienopyrazolyls (e.g., 1H-thieno[2,3-c]pyrazolyl, benzothiaphen-yl, thienopyridyl (e.g., thieno[3,2-b]pyridyl or thieno[3,2-c]pyridyl), thienopyridazinyl (e.g., thieno[3,2-c]pyridazinyl), tetrahydrothienopyridyl (e.g., 4,5,6,7-tetrahydrothieno[3,2-c]pyridine), and pyrrolopyridines (e.g., 1H-pyrrolyl[2,3-c]pyridine). In some embodiments, A is unsubstituted. In some embodiments, A is substituted with 1, 2, or 3 substituent groups as described herein. In some embodiments, A is substituted with one or two halogen groups or an unsubstituted phenyl group.

In some embodiments, A is

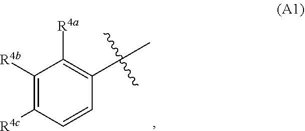

wherein
R^{4a}, R^{4b}, and R^{4c} are independently H, halo, aryl, heteroaryl, CH₂OR¹², OR¹², NHR¹² or CH₂R¹³;
R¹² is H, aryl optionally substituted with R¹⁴, or C₁-C₂ alkyl optionally substituted with R¹⁵;
R¹³ is a heterocycloalkyl;
R¹⁴ is H or halo; and
R¹⁵ is cycloalkyl or aryl optionally substituted with halo.

In some embodiments, each of R^{4a} and R^{4c} is H. In some embodiments, R^{4b} is halogen. In some embodiments, R^{4b} is chloro.

In some embodiments, R¹³ is pyrrolidine.

In some embodiments, A is

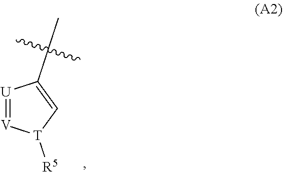

wherein
U, V, and T are independently CH, or N;
R⁵ is C₁-C₄ alkyl optionally substituted with R¹⁶; aryl optionally substituted with H, halo, CF₃; heterocycloalkyl optionally substituted with COOR¹⁷; or heteroaryl optionally substituted with COOR¹⁷;
R¹⁶ is H, aryl optionally substituted with halo, or heterocycloalkyl; and
R⁷ is t-butyl.

In some embodiments, one of U, V, and T is N, and two are CH.

In some embodiments, two of U, V, and T is N, and one is CH.

In some embodiments, R⁵ is optionally substituted phenyl. In some embodiments, R⁵ is unsubstituted phenyl.

In some embodiments, A is

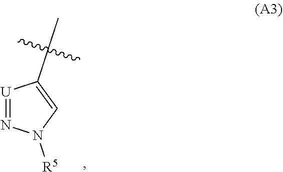

wherein
U is CH or N;
R⁵ is C₁-C₄ alkyl optionally substituted with R¹⁶; aryl optionally substituted with H, halo, or CF₃; heterocycloalkyl optionally substituted with COOR¹⁷; or heteroaryl optionally substituted with COOR¹⁷;
R¹⁶ is H, aryl optionally substituted with halo, or heterocycloalkyl; and
R¹⁷ is t-butyl.

In some embodiments, U is CH.

In some embodiments, U is N.

In some embodiments, R⁵ is optionally substituted phenyl. In some embodiments, R⁵ is unsubstituted phenyl.

In some embodiments, A is

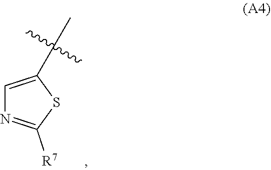

wherein
R⁷ is H, optionally substituted C₁-C₃ alkyl, or optionally substituted phenyl.

In some embodiments, R⁷ is H.

In some embodiments, R⁷ is optionally substituted C₁-C₃ alkyl. In some embodiments, R⁷ is unsubstituted C₁-C₃ alkyl.

In some embodiments, R⁷ is optionally substituted phenyl. In some embodiments, R⁷ is unsubstituted phenyl.

In some embodiments, A is

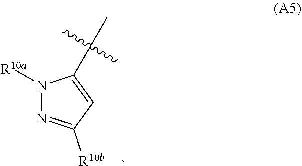

wherein
R^{10a} is H or optionally substituted C₁-C₃ alkyl; and
R^{10b} is H or optionally substituted thiazole.

In some embodiments, R^{10a} is H.

In some embodiments, R^{10a} is optionally substituted C₁-C₃ alkyl. In some embodiments, R^{10a} is unsubstituted C₁-C₃ alkyl.

In some embodiments, R^{10b} is H.

In some embodiments, R^{10b} is optionally substituted thiazole. In some embodiments, R^{10b} is unsubstituted thiazole.

In some embodiments, R^{10a} and R^{10b} are both H.

In some embodiments, R^{10a} is H, and R^{10b} is optionally substituted thiazole.

In some embodiments, A is

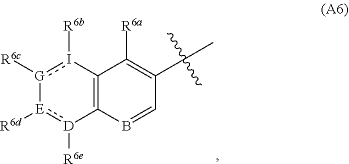

wherein
B, D, E, G, and I are independently C, CH, or N;
R^{6a}, R^{6b}, R^{6c}, and R^{6d} are independently H, C₁-C₃ alkyl, halo, OR¹⁸, or NHR¹⁹,
R^{6e} is H or ═O;
R¹⁸ is H, aryl optionally substituted with halo, or C₁-C₃ alkyl optionally substituted with R²⁰.
R¹⁹ is SO₂CH₃;
R²⁰ is aryl optionally substituted with halo; and
------ is an optional bond.

In some embodiments, ------- is not present, and [symbol] represents a single bond. In such embodiments, the valences of D, E, G, and/or I may be completed with a hydrogen as required. In some embodiments, no ------- is present.

In some embodiments, ------- is present, and [symbol] represents a double bond. In some embodiments, each ------- is present.

In some embodiments, at least one of B, D, E, G, and I is N.

In some embodiments, no more than two of B, D, E, G, and I are N.

In some embodiments, each of D, E, G, and I is C or CH.

In some embodiments, each of R^{6a}, R^{6b}, R^{6c}, and R^{6d} is H.

In some embodiments, A is

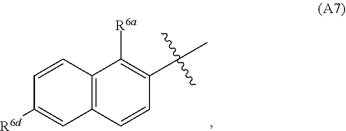

wherein
R^{6a} is H or methyl;
R^{6d} is H, OR¹⁸, or NHR¹⁹,
R¹⁸ is H, aryl optionally substituted with halo, or C₁-C₃ alkyl optionally substituted with R²⁰;
R¹⁹ is SO₂CH₃; and
R²¹ is aryl optionally substituted with halo.

In some embodiments, each of R^{6a} and R^{6d} is H.

In some embodiments, A is

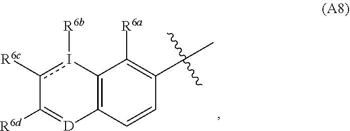

wherein
I is C, CH, or N;
D is CH or N;
R^{6a} is H or halo;
R^{6b} is H or optionally substituted C₁-C₃ alkyl;
R^{6c} is H, ═O or optionally substituted C₁-C₃ alkyl;
R^{6d} is H or C₁-C₃ alkyl; and
----- is an optional bond.

In some embodiments, ------- is not present, and [symbol] represents a single bond. In such embodiments, the valences of I and CR^{6c} may be completed with a hydrogen as required.

In some embodiments, ------- is present, and [symbol] represents a double bond.

In some embodiments, ------- is absent and I is N. In some embodiments, D is N. In some embodiments, D is CH.

In some embodiments, D is N. In some embodiments, ------- is absent and I is N. In some embodiments, I is C or CH.

In some embodiments, D is CH and I is C or CH.

In some embodiments, each of R^{6a}, R^{6b}, R^{6c}, and R^{6d} is H.

In some embodiments, A is

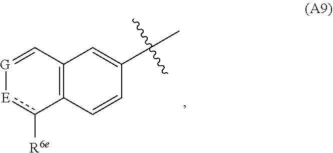

wherein
E is CH, CH₂, or N;
G is CH or N;
R^{6e} is H or ═O; and
----- is an optional bond.

In some embodiments, ------- is not present, and [symbol] represents a single bond. In such embodiments, the valences of E and CR^{6e} may be completed with a hydrogen as required.

In some embodiments, ------- is present, and [symbol] represents a double bond.

In some embodiments, G is N. In some embodiments, E is CH₂ or CH. In some embodiments, E is N.

In some embodiments, G is N and E is CH or N.

In some embodiments, E is N. In some embodiments, G is CH. In some embodiments, G is N.

In some embodiments, G is CH, and E is CH or CH₂.

In some embodiments, R^{6e} is H.

In some embodiments, A is

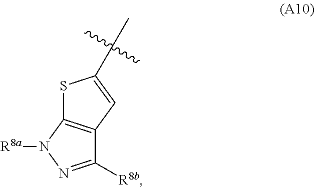

wherein
R^{8a} and R^{8b} are independently H or optionally substituted C₁-C₃ alkyl.

In some embodiments, R^{8a} is H.

In some embodiments, R^{8b} is H.

In some embodiments, R^{8a} is optionally substituted C₁-C₃ alkyl. In some embodiments, R^{8a} is unsubstituted C₁-C₃ alkyl.

In some embodiments, R^{8b} is optionally substituted C₁-C₃ alkyl. In some embodiments, R^{8b} is unsubstituted C₁-C₃ alkyl.

In some embodiments, R^{8a} and R^{8b} are both H.

In some embodiments, A is

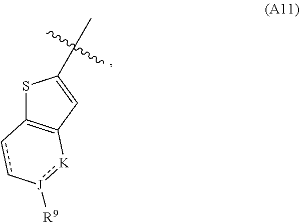

wherein
J is C, CH, or N;
K is CH, CH₂, N, or NH;
R⁹ is H, halo, optionally substituted C₁-C₄ alkyl, or CO₂R²¹;
R²¹ is H or optionally substituted C₁-C₅ alkyl (e.g., t-butyl); and
------ is an optional bond.

In some embodiments, ------- is not present, and [symbol] represents a single bond. In some embodiments, no ------- is present.

In some embodiments, ------- is present, and [symbol] represents a double bond. In some embodiments, each ------- is present.

In some embodiments, ------- is absent and J is N. In some embodiments, K is CH₂ or CH. In some embodiments, ------- is present and K is N.

In some embodiments, J is C or CH. In some embodiments, J is N.

In some embodiments, K is N and J is C.

In some embodiments, K is CH₂ and J is N.

In some embodiments, R⁹ is H.

In some embodiments, R⁹ is halogen.

In some embodiments, R⁹ is optionally substituted C₁-C₄ alkyl. In some embodiments, R⁹ is unsubstituted C₁-C₄ alkyl.

In some embodiments, A is

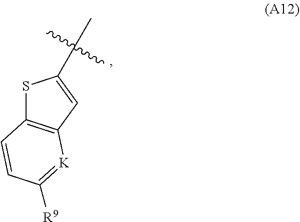

wherein
K is CH or N; and
R⁹ is H, halo, or optionally substituted C₁-C₄ alkyl.

In some embodiments, K is CH.

In some embodiments, K is N.

In some embodiments, R⁹ is H.

In some embodiments, R⁹ is halogen.

In some embodiments, R⁹ is optionally substituted C₁-C₄ alkyl. In some embodiments, R⁹ is unsubstituted C₁-C₄ alkyl.

In some embodiments, A is

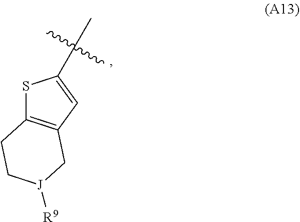

wherein
J is CH or N;
R⁹ is H or CO₂R²¹; and
R²¹ is optionally substituted C₁-C₅ alkyl (e.g., t-butyl).

In some embodiments, J is CH.

In some embodiments, J is N.

In some embodiments, R⁹ is H.

In some embodiments, R⁹ is CO₂R²¹.

In some embodiments, R²¹ is t-butyl.

In some embodiments, A is

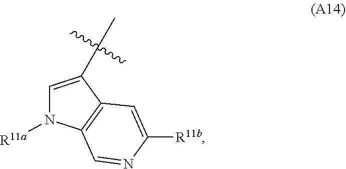

wherein
R^{11a} and R^{11b} are independently H, optionally substituted C₁-C₃ alkyl, or optionally substituted C₁-C₃ alkoxy.

In some embodiments, R^{11a} is H.

In some embodiments, R^{11b} is H.

In some embodiments, R^{11a} is optionally substituted C₁-C₃ alkyl. In some embodiments, R^{11a} is unsubstituted C₁-C₃ alkyl.

In some embodiments, R^{11b} is optionally substituted C₁-C₃ alkyl. In some embodiments, R^{11b} is unsubstituted C₁-C₃ alkyl.

In some embodiments, R^{11a} is optionally substituted C₁-C₃ alkoxy. In some embodiments, R^{11a} is unsubstituted C₁-C₃ alkoxy.

In some embodiments, R^{11b} is optionally substituted C₁-C₃ alkoxy. In some embodiments, R^{11b} is unsubstituted C₁-C₃ alkoxy.

In some embodiments, R^{11a} and R^{b} are both H.

In some embodiments, A is any one of substructures A1-A14.

In some embodiments, A is

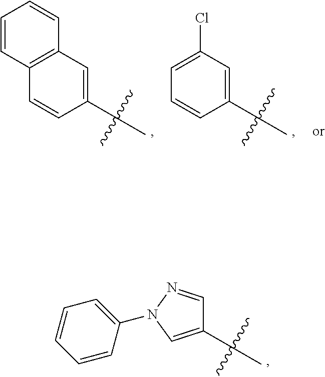 [SMILES: *c1ccc2ccccc2c1 ; *c1cccc(Cl)c1 ; *c1cnn(-c2ccccc2)c1]

### Exemplary Compounds

In some embodiments, the PHD1 inhibitor compound is any one of Compounds 1-17, or a pharmaceutically acceptable salt thereof.

| Cmpd. | |
|---|---|
| No. | Structure |
| 1 | 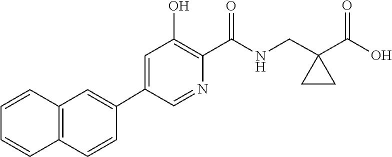 [SMILES: O=C(NCC1(C(=O)O)CC1)c1ncc(-c2ccc3ccccc3c2)cc1O] |
| 2 | 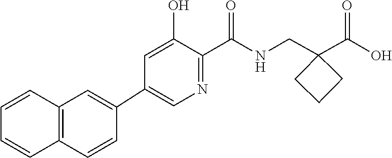 [SMILES: O=C(NCC1(C(=O)O)CCC1)c1ncc(-c2ccc3ccccc3c2)cc1O] |
| 3 | 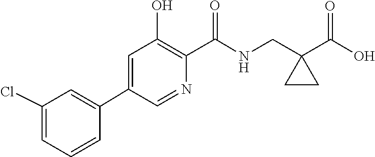 [SMILES: O=C(NCC1(C(=O)O)CC1)c1ncc(-c2cccc(Cl)c2)cc1O] |
| 4 | 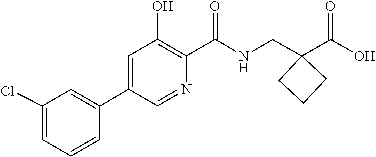 [SMILES: O=C(NCC1(C(=O)O)CCC1)c1ncc(-c2cccc(Cl)c2)cc1O] |
| 5 | 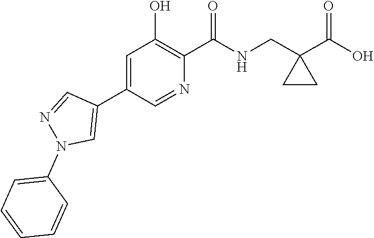 |
| 6 | 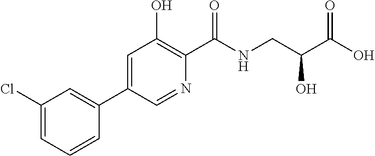 [SMILES: O=C(NC[C@H](O)C(=O)O)c1ncc(-c2cccc(Cl)c2)cc1O] |
| 7 | 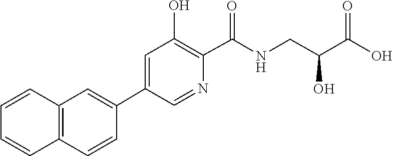 [SMILES: O=C(NC[C@H](O)C(=O)O)c1ncc(-c2ccc3ccccc3c2)cc1O] |
| 8 | 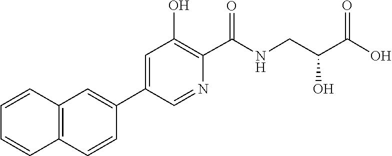 [SMILES: O=C(NC[C@@H](O)C(=O)O)c1ncc(-c2ccc3ccccc3c2)cc1O] |
| 9 | 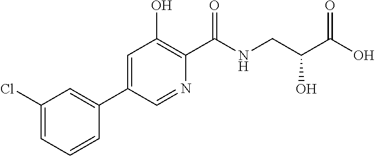 [SMILES: O=C(NC[C@@H](O)C(=O)O)c1ncc(-c2cccc(Cl)c2)cc1O] |
| 10 | 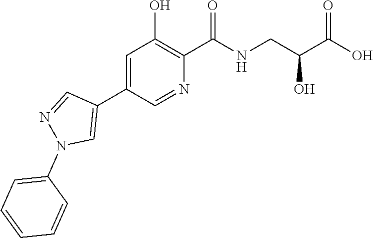 |
| 11 | 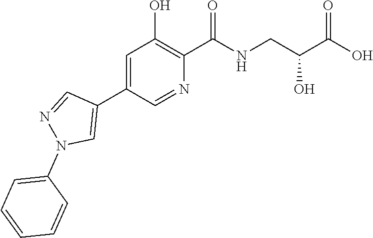 |
| 12 | 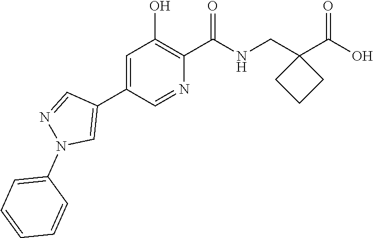 |
| 13 | 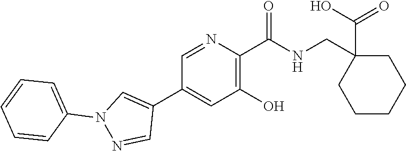 [SMILES: O=C(NCC1(C(=O)O)CCCCC1)c1ncc(-c2cnn(-c3ccccc3)c2)cc1O] |
| 14 | 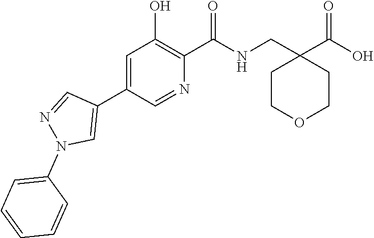 |
| 15 | 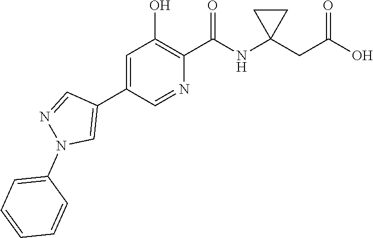 |
| 16 | 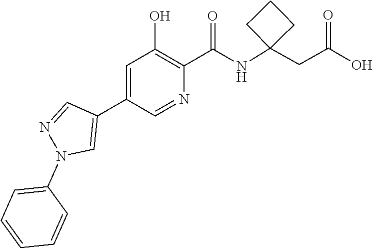 |
| 17 | 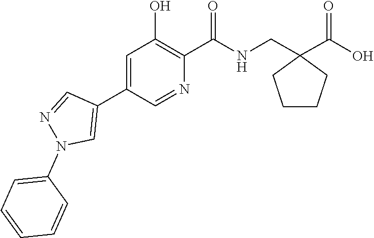 |

In some embodiments, a PHD1 inhibitor compound is Compound 1. In some embodiments, a PHD1 inhibitor compound is Compound 2. In some embodiments, a PHD1 inhibitor compound is Compound 3. In some embodiments, a PHD1 inhibitor compound is Compound 4. In some embodiments, a PHD1 inhibitor compound is Compound 5. In some embodiments, a PHD1 inhibitor compound is Compound 6. In some embodiments, a PHD1 inhibitor compound is Compound 7. In some embodiments, a PHD1 inhibitor compound is Compound 8. In some embodiments, a PHD1 inhibitor compound is Compound 9. In some embodiments, a PHD1 inhibitor compound is Compound 10. In some embodiments, a PHD1 inhibitor compound is Compound 11. In some embodiments, a PHD1 inhibitor compound is Compound 12. In some embodiments, a PHD1 inhibitor compound is Compound 13. In some embodiments, a PHD1 inhibitor compound is Compound 14. In some embodiments, a PHD1 inhibitor compound is Compound 15. In some embodiments, a PHD1 inhibitor compound is Compound 16. In some embodiments, a PHD1 inhibitor compound is Compound 17. In some embodiments, a PHD1 inhibitor compound is a pharmaceutically acceptable salt of any of these compounds.

### Isotopologues

It should be understood that in the compounds described herein (e.g., a compound of any one of Formulas I-IV such as any one of compounds 1-17), the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of the compounds described herein (e.g., a compound of any one of Formulas I-IV such as any one of compounds 1-17). For example, different isotopic forms of hydrogen (H) include protium (¹H), deuterium (²H), and tritium (³H). Protium is the predominant hydrogen isotope found in nature.

In some embodiments, one or more of the hydrogens of the compounds described herein (e.g., a compound of any one of Formulas I-IV such as any one of compounds 1-17) is replaced by a deuterium. Enriching for deuterium may afford certain therapeutic advantages, such as increasing in vivo half-life or reducing dosage requirements, or may provide a compound useful as a standard for characterization of biological samples. In some embodiments, one or more of the hydrogens of the compounds described herein (e.g., a compound of any one of Formulas I-IV such as any one of compounds 1-17) is replaced by tritium. Tritium is radioactive and may therefore provide for a radiolabeled compound, useful as a tracer in metabolic or kinetic studies.

Isotopic-enrichment of compounds disclosed herein (e.g., a compound of any one of Formulas I-IV such as any one of compounds 1-17), may be achieved without undue experimentation by conventional techniques well known to those skilled in the art or by processes analogous to those described in the Schemes and Examples herein using appropriate isotopically-enriched reagents and/or intermediates.

The term “isotopologue” refers to a species that has the same chemical structure and formula as a specific compound provided herein, with the exception of the positions of isotopic substitution and/or level of isotopic enrichment at one or more positions, e.g., hydrogen vs. deuterium. Thus, the term “compound,” as used herein, encompasses a collection of molecules having identical chemical structure, but also having isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound provided depends upon a number of factors including, but not limited to, the isotopic purity of deuterated reagents used to make the compound and the efficiency of incorporation of deuterium in the various synthesis steps used to prepare the compound.

When a position is designated as “H” or “hydrogen”, the position is understood to have hydrogen at its natural abundance isotopic composition. When a position is designated as “2H” or “deuterium”, the position is understood to have deuterium at an abundance that is at least 3340 times greater than the natural abundance of deuterium, which is 0.015% (i.e., the term “²H” or “deuterium” indicates at least 50.1% incorporation of deuterium).

In embodiments, a compound provided herein may have an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Synthesis of Compounds of the Inventions

The compounds described herein (e.g., a compound of any one of Formulas I-IV such as any one of compounds 1-17) can be prepared according to methods known in the art, including the exemplary syntheses of the Examples provided herein, such as the syntheses shown in Schemes A and B.

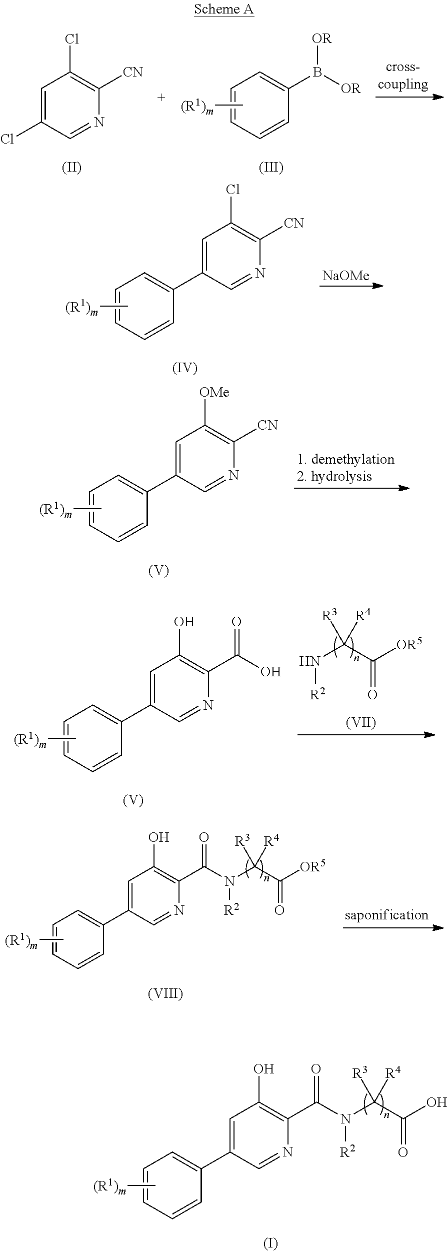

Compounds of Formula (I) are prepared according to Scheme A using commercially available materials. The cross-coupling of (II) and (III) using a palladium catalyst yields the biaryl compounds of formula (IV). Nucleophilic aromatic substitution of (IV) with sodium methoxide at elevated temperatures furnishes the compounds of formula (V). Next, compounds (V) are subjected to a demethylation reagent such as HBr (aq.) or BBr₃ at elevated temperatures followed by hydrolysis conditions using hydroxide bases, such as NaOH and KOH. The amide compounds (VIII) are synthesized using (VI) and a coupling reactant such as CDI, EDCI, or (COCl)₂, followed by the addition of amino acids (VII) and an amine base, such as DIPEA or Et₃N. Lastly, the ester compounds of formula (VIII) are saponified using a suitable base such as NaOH, LiOH, and KOH in a combination of solvents such as THF or dioxane and water.

Alternatively, compounds of Formula (I) are prepared according to Scheme B using commercially available starting materials. The ester of formula (IX) are reacted with amino acids (VII) and a base such as DIPEA or K₂CO₃ in a high boiling solvent such as dioxane or DMF at elevated temperatures to provide compounds of formula (X). The cross-coupling of (X) and (XI) using a palladium catalyst yields the biaryl compounds of formula (VIII). Similar to Scheme A, the ester compounds of formula (VIII) are saponified using a suitable base such as NaOH, LiOH and KOH in a combination of solvents such as THF or dioxane and water.

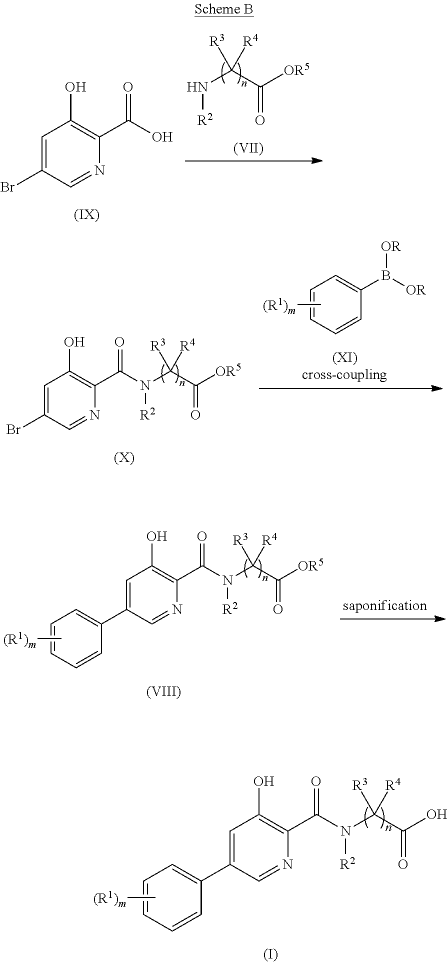

### Compositions and Methods

The invention provides for use of a compound of any one of Formulas (I)-(IV), or a pharmaceutically acceptable salt thereof, for the manufacture of a medicament for use in treating various conditions or disorders as described herein. In one embodiment, a pharmaceutical composition is provided comprising at least one compound of any one of Formulas (I)-(IV), or a pharmaceutically acceptable salt thereof, and a pharmaceutically acceptable excipient or carrier. In various embodiments, the medicament or pharmaceutical composition can further comprise or be used in combination with at least one additional therapeutic agent.

The compounds of the present invention, or medicaments or compositions comprising the compounds, can be used to inhibit PHD 1 activity selectively over other isoforms, for example, PHD2 enzyme. Selective inhibition of PHD 1 may be of particular benefit in treating ischemia reperfusion injury (including but not limited to stroke, myocardial infarction, and acute kidney injury) inflammatory bowel disease, cancer (including colorectal cancer) and liver disease. In one embodiment, the method of the invention comprises administering to a patient in need a therapeutically effective amount of a compound of any one of Formulas (I)-(IV), or a pharmaceutically acceptable salt thereof, or a pharmaceutical composition comprising one or more compounds of any one of Formulas (I)-(IV).

In some embodiments, compounds described herein are useful for treating or preventing a non-anemia disease.

The invention is also directed to a method of inhibiting the activity of PHD1. The PHD1 enzyme is selectively inhibited over other PHD isoforms, for example, PHD2 enzyme. In one embodiment, the method comprises contacting PHD1 with an effective amount of one or more compounds selected from the group comprising compounds of any one of Formulas (I)-(IV), or a pharmaceutically acceptable salt thereof.

In still other embodiments, the compounds disclosed herein (e.g., a compound of any one of Formulas (I)-(IV) such as any one of compounds 1-17), or a pharmaceutically acceptable salt thereof, are useful for the treatment or prevention of ischemia reperfusion injury. These include but are not limited to stroke, myocardial infarction, and acute kidney injury).

In other embodiments, the compounds disclosed herein (e.g., a compound of any one of Formulas (I)-(IV) such as any one of compounds 1-17), or a pharmaceutically acceptable salt thereof, are useful in the treatment of inflammatory bowel disease.

In other embodiments, the compounds disclosed herein (e.g., a compound of any one of Formulas (I)-(IV) such as any one of compounds 1-17), or a pharmaceutically acceptable salt thereof, are useful in the treatment of cancers, such as colorectal cancer.

In yet other embodiments, the compounds disclosed herein (e.g., a compound of any one of Formulas (I)-(IV) such as any one of compounds 1-17), or a pharmaceutically acceptable salt thereof, are useful in the treatment of liver disease.

In other embodiments, the compounds disclosed herein (e.g., a compound of any one of Formulas (I)-(IV) such as any one of compounds 1-17), or a pharmaceutically acceptable salt thereof, are useful in the treatment of retinopathy of prematurity (ROP).

In addition, the compounds disclosed herein (e.g., a compound of any one of Formulas (I)-(IV) such as any one of compounds 1-17), or a pharmaceutically acceptable salt thereof, may be used in combination with additional active ingredients in the treatment of the above conditions. The additional compounds may be co-administered separately with the compounds disclosed herein (e.g., a compound of any one of Formulas (I)-(IV) such as any one of compounds 1-17), or a pharmaceutically acceptable salt thereof, or included with an additional active ingredient in a pharmaceutical composition according to the invention. In an exemplary embodiment, additional active ingredients are those that are known or discovered to be effective in the treatment of conditions, disorders, or diseases mediated by PHD enzyme or that are active against another targets associated with the particular condition, disorder, or disease, such as an alternate PHD modulator. The combination may serve to increase efficacy (e.g., by including in the combination a compound potentiating the potency or effectiveness of a compound according to the invention), decrease one or more side effects, or decrease the required dose of the compound according to the invention.

The compounds of the invention are used, alone or in combination with one or more other active ingredients, to formulate pharmaceutical compositions of the invention. A pharmaceutical composition of the invention comprises: (a) an effective amount of the compounds disclosed herein (e.g., a compound of any one of Formulas (I)-(IV) such as any one of compounds 1-17), or a pharmaceutically acceptable salt, pharmaceutically acceptable prodrug, or pharmaceutically active metabolite thereof; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols. Suitable excipients may also include antioxidants. Such antioxidants may be used in a pharmaceutical composition or in a storage medium to prolong the shelf-life of the drug product.

### Pharmaceutical Formulations and Routes of Administration

The compounds (or a pharmaceutically acceptable salt thereof) and compositions of the present invention can be delivered directly or in pharmaceutical compositions or medicaments along with suitable carriers or excipients, as is well known in the art. Present methods of treatment can comprise administration of an effective amount of a compound of the invention to a subject in need. In a preferred embodiment, the subject is a mammalian subject, and in a most preferred embodiment, the subject is a human subject.

An effective amount of such compound, composition, or medicament can readily be determined by routine experimentation, as can the most effective and convenient route of administration, and the most appropriate formulation. Various formulations and drug delivery systems are available in the art. See, e.g., Gennaro, A. R., ed. (1995) Remington's Pharmaceutical Sciences, supra.

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

Pharmaceutical dosage forms of a compound of the invention, or a pharmaceutically acceptable salt thereof, may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art and include those listed in various pharmacopoeias. See, e.g., the U.S. Pharmacopeia (USP), Japanese Pharmacopoeia (JP), European Pharmacopoeia (EP), and British pharmacopeia (BP); the U.S. Food and Drug.

Administration (www.fda.gov) Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott NY; etc.) [0149] Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms, and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e., dextrose, sucrose, lactose, etc.), talc, tragacanth mucilage, vegetable oils (hydrogenated), and waxes. Ethanol and water may serve as granulation aides. In certain instances, coating of tablets with, for example, a taste- masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention, or a pharmaceutically acceptable salt thereof, can be administered topically, such as through a skin patch, a semi-solid, or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention, or a pharmaceutically acceptable salt thereof, are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compounds (or pharmaceutically acceptable salts thereof) and compositions formulated for parenteral administration by injection are usually sterile and can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the compounds of the invention, or a pharmaceutically acceptable salt thereof, are well-known in the art and include water-based solutions containing a base, such as, for example, sodium hydroxide, to form an ionized compound; sucrose or sodium chloride as a tonicity agent; and a buffer, for example, a buffer that contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

A therapeutically effective dose can be estimated initially using a variety of techniques well- known in the art. Initial doses used in animal studies may be based on effective concentrations established in cell culture assays. Dosage ranges appropriate for human subjects can be determined, for example, using data obtained from animal studies and cell culture assays. In some certain embodiments, a compound of the disclosure is formulated for oral administration. An exemplary dose of a compound of the disclosure in a pharmaceutical formulation for oral administration is from about 0.5 to about 10 mg/kg body weight of subject. In some embodiments, a pharmaceutical formulation comprises from about 0.7 to about 5.0 mg/kg body weight of subject, or alternatively, from about 1.0 to about 2.5 mg/kg body weight of subject. A typical dosing regimen for oral administration would be administration of the pharmaceutical formulation for oral administration three times per week, two times per week, once per week or daily.

An effective amount or a therapeutically effective amount or dose of an agent, e.g., a compound of the invention, or a pharmaceutically acceptable salt thereof, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The effective amount or therapeutically effective amount is the amount of the compound, or a pharmaceutically acceptable salt thereof, or pharmaceutical composition that will elicit the biological or medical response of a tissue, system, animal or human that is being sought by the researcher, veterinarian, medical doctor or other clinician. Dosages particularly fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of compound, or a pharmaceutically acceptable salt thereof, or composition administered may be dependent on a variety of factors, including the sex, age, and weight of the subject being treated, the severity of the affliction, the manner of administration, and the judgment of the prescribing physician.

The present compounds, or a pharmaceutically acceptable salt thereof, and compositions may, if desired, be presented in a pack or dispenser device containing one or more unit dosage forms containing the active ingredient. Such a pack or device may, for example, comprise metal or plastic foil, such as a blister pack; or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein and are specifically contemplated.

### EXEMPLIFICATION

Abbreviations and acronyms used herein including the following:

| Term | Acronym |
|---|---|
| 1,1′-Carbonyldiimidazole | CDI |
| 1-(3-Dimethylaminopropyl)-3- | EDCI |
| ethylcarbodiimide hydrochloride | |
| Acetyl | Ac |
| Aqueous | aq. |
| Broad singlet | brs |
| Dichloromethane | DCM |
| Dimethylsulfoxide | DMSO |
| Doublet | d |
| Electrospray ionization | ESI |
| Ethyl acetate | EtOAc |
| Gram | g |
| Hexanes | Hex |
| High performance liquid chromatography | HPLC |
| Hour | hr |
| Hydrobromic acid | HBr |
| Isopropyl | i-Pr |
| Liquid chromatography-mass spectrometry | LCMS |
| Megahertz | MHz |
| Milligram | mg |
| Milliliter | mL |
| Minute | min |
| Multiplet | m |
| n-Butyl | n-Bu |
| N,N-Diisopropylethylamine | DIPEA |
| N,N-Dimethylformamide | DMF |
| Nuclear magnetic resonance | NMR |
| Palladium on carbon | Pd/C |
| Pentet | p |
| Phenyl | Ph |
| Potassium hydroxide | KOH |
| Quartet | q |
| Room temperature | RT |
| Singlet | s |
| Sodium hydroxide | NaOH |
| Sodium methoxide | NaOMe |
| Tetrabutylammonium fluoride | TBAF |
| Tetrahydrofuran | THF |
| Thin layer chromatography | TLC |
| Tosyl | Ts |
| Triethylamine | TEA |
| Trimethylsilylacetylene | TMSA |
| Triplet | t |

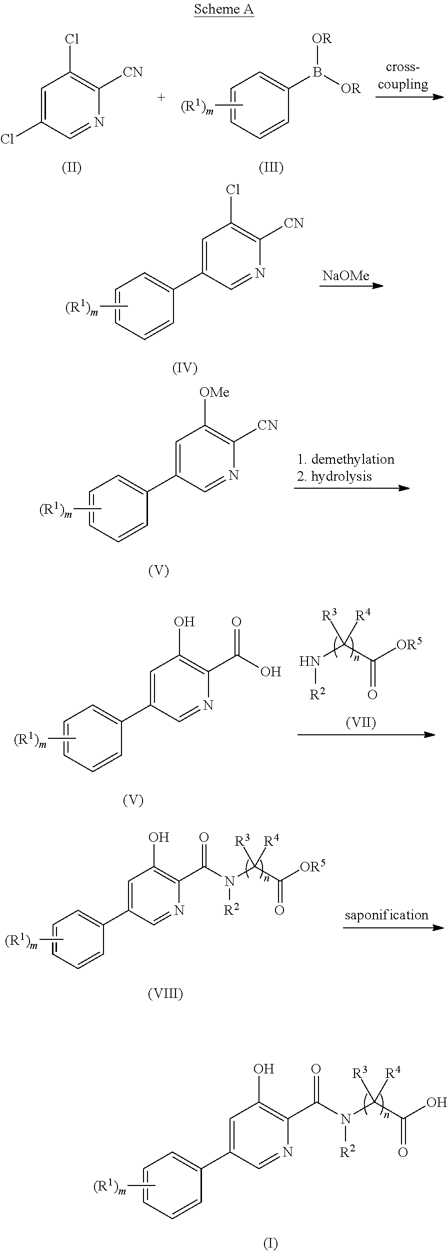

Compounds of Formula (I) are prepared according to Scheme A using commercially available materials. The cross-coupling of (II) and (III) using a palladium catalyst yields the biaryl compounds of formula (IV). Nucleophilic aromatic substitution of (IV) with sodium methoxide at elevated temperatures furnishes the compounds of formula (V). Next, compounds (V) are subjected to a demethylation reagent such as HBr (aq.) or BBr₃ at elevated temperatures followed by hydrolysis conditions using hydroxide bases, such as NaOH and KOH. The amide compounds (VIII) are synthesized using (VI) and a coupling reactant such as CDI, EDCI, or (COCl)₂, followed by the addition of amino acids (VII) and an amine base, such as DIPEA or Et₃N. Lastly, the ester compounds of formula (VIII) are saponified using a suitable base such as NaOH, LiOH, and KOH in a combination of solvents such as THF or dioxane and water.

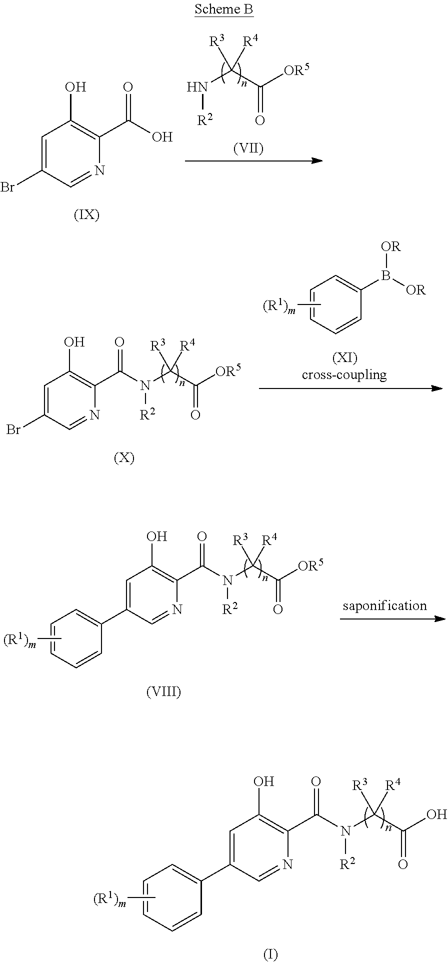

Alternatively, compounds of Formula (I) are prepared according to Scheme B using commercially available starting materials. The ester of formula (IX) are reacted with amino acids (VII) and a base such as DIPEA or K₂CO₃ in a high boiling solvent such as dioxane or DMF at elevated temperatures to provide compounds of formula (X). The cross-coupling of (X) and (XI) using a palladium catalyst yields the biaryl compounds of formula (VIII). Similar to Scheme A, the ester compounds of formula (VIII) are saponified using a suitable base such as NaOH, LiOH, and KOH in a combination of solvents such as THF or dioxane and water.

### Synthesis for Exemplary Compounds

### Example 1: Preparation of Compound 1

### Methyl 3-(benzyloxy)-5-bromopicolinate

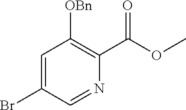 [SMILES: COC(=O)c1ncc(Br)cc1OCc1ccccc1]

To a solution of methyl 5-bromo-3-hydroxypicolinate (2.00 g, 8.60 mmol) in DMF (20 ml) was added in benzyl bromide (1.18 g, 10.30 mmol) and Cs₂CO₃ (2.80 g, 8.60 mmol) in one portion. After addition, the mixture was stirred at rt overnight. After the reaction was completed as indicated by TLC, the mixture was diluted with water (100 mL) and extracted with EtOAc (50 mL×3). The combined organic phased were dried with Na₂SO₄, filtered and concentrated. The residue was slurried with hexane (10 mL) for 2 hrs and filtered to get the desired product (2.31 g) as a white solid. LC-MS (ESI+): m/z 322 (M+H)⁺; ¹H-NMR (300 MHz, CDCl₃) δ 8.35 (d, J=1.8 Hz, 1H), 7.55 (d, J=1.8 Hz, 1H), 7.35-7.48 (m, 5H), 5.21 (s, 2H), 3.98 (s, 3H).

### Methyl 3-(benzyloxy)-5-(naphthalen-2-yl)picolinate

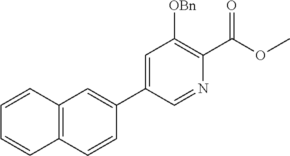 [SMILES: COC(=O)c1ncc(-c2ccc3ccccc3c2)cc1OCc1ccccc1]

Under nitrogen protection, a solution of methyl 3-(benzyloxy)-5-bromopicolinate (1.23 g, 3.83 mmol) in DMF (20 ml) was added in naphthalen-2-ylboronic acid (0.98 g, 5.75 mmol), K₂CO₃ (1.59 g, 11.50 mmol) and Pd(PPh₃)₄ (0.31 g, 0.27 mmol) in one portion. The mixture was stirred at 80° C. overnight. After the reaction was completed as indicated by TLC, the mixture was diluted with water (100 mL) and extracted with EtOAc (50 mL×3). The combined organic phased were dried with Na₂SO₄, filtered and concentrated. The residue was purified by flash silica chromatography (EA:PE=1:20 to 1:5) to give the desired product (1.24 g) as white solid. LC-MS (ESI+): m/z 370 (M+H)⁺; ¹H-NMR (300 MHz, CDCl₃) δ 8.64 (s, 1H), 7.88-8.00 (m, 4H), 7.63-7.66 (m, 2H), 7.53-7.59 (m, 4H), 7.33-7.46 (m, 3H), 5.34 (s, 2H), 4.03 (s, 3H).

### Methyl 3-hydroxy-5-(naphthalen-2-yl)picolinate

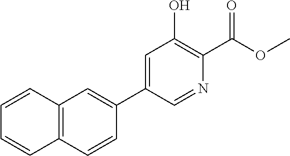 [SMILES: COC(=O)c1ncc(-c2ccc3ccccc3c2)cc1O]

A suspension of methyl 3-(benzyloxy)-5-(naphthalen-2-yl)picolinate (1.24 g, 2.50 mmol) and Pd/C (124 mg) in MeOH (20 mL) was stirred under hydrogen atmosphere at rt overnight. After the reaction was completed as indicated by TLC, the suspension was filtered through a package of Celite and the filtered cake was washed with MeOH (10 mL). The combined filtrate was concentrated to dryness to give the desired product (710 mg) as oil. LC-MS (ESI+): m/z 280 (M+H⁺), ¹H NMR (300 MHz, CDCl₃) δ 10.74 (s, 1H), 8.65 (d, J=2.1 Hz, 1H), 8.10 (s, 1H), 7.88-7.99 (m, 3H), 7.68-7.74 (m, 2H), 7.54-7.58 (m, 2H), 4.10 (s, 3H).

### 3-Hydroxy-5-(naphthalen-2-yl)picolinic acid

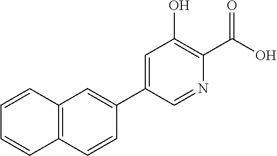 [SMILES: O=C(O)c1ncc(-c2ccc3ccccc3c2)cc1O]

To a suspension of methyl 3-hydroxy-5-(naphthalen-2-yl)picolinate (0.57 g, 2.04 mmol) in THF (10 mL) and water (4 mL) was added in KOH (1.71 g, 30.6 mmol) in one portion. The mixture was stirred at 110° C. for 6 hrs. After the reaction was completed as indicated by HPLC, the suspension was diluted with water (10 mL) and adjusted the pH to 3. A large amount of solid was precipitated. The suspension was filtered and dried to give the desired product (500 mg) as white solid. LC-MS (ESI+): m/z 266 (M+H)⁺, ¹H NMR (300 MHz, DMSO-d6) δ 8.60 (s, 1H), 8.46 (s, 1H), 7.96-8.09 (m, 5H), 7.59-7.62 (m, 2H).

### ethyl 1-((3-hydroxy-5-(naphthalen-2-yl)picolinamido)methyl)cyclopropane-1-carboxylate

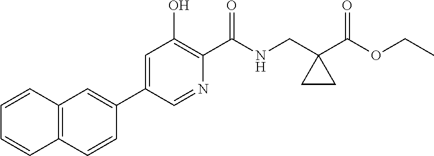 [SMILES: CCOC(=O)C1(CNC(=O)c2ncc(-c3ccc4ccccc4c3)cc2O)CC1]

To a solution of 3-hydroxy-5-(naphthalen-2-yl)picolinic acid (0.10 g, 0.38 mmol) in DMF (5 mL) was added in ethyl 1-(aminomethyl)cyclopropane-1-carboxylate (54.40 mg, 0.38 mmol), PyBOP (0.49 g, 0.95 mmol) and TEA (76.90 mg, 0.76 mmol) in one portion. The mixture was stirred at rt for 4.5 hrs. After the reaction was completed as indicated by TLC, the mixture was diluted with water (100 mL) and extracted with EtOAc (50 mL×3). The combined organic phased were washed with water (20 mL), dried with Na₂SO₄, filtered and concentrated. The residue was purified by flash silica chromatography (EA:PE=1:10) to give the desired product (55 mg) as oil. ¹H NMR (300 MHz, CDCl₃) δ 12.26 (s, 1H), 8.66 (s, 1H), 8.46 (s, 1H), 8.08 (s, 1H), 7.89-7.98 (m, 3H), 7.72 (d, J=8.1 Hz, 1H), 7.55-7.61 (m, 3H), 4.18-4.25 (q, J=7.2 Hz, 2H), 3.64 (d, J=6.3 Hz, 2H), 1.32-1.37 (m, 3H), 1.26-1.29 (m, 2H), 0.86-0.99 (m, 2H).

### 1-((3-hydroxy-5-(naphthalen-2-yl)picolinamido)methyl)cyclopropane-1-carboxylic acid

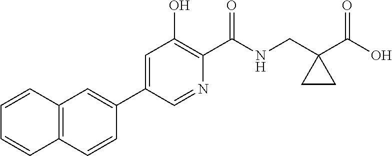 [SMILES: O=C(NCC1(C(=O)O)CC1)c1ncc(-c2ccc3ccccc3c2)cc1O]

To a solution of ethyl 1-((3-hydroxy-5-(naphthalen-2-yl)picolinamido)methyl) cyclopropane-1-carboxylate (50.0 mg, 0.13 mmol) in THF (5 mL) and water (1.25 mL) was added in LiOH (43.00 mg, 1.02 mmol) in one portion. The mixture was stirred at 50° C. overnight. After the reaction was completed as indicated by TLC, the solution was diluted with water (10 mL) and adjusted the pH to 3. A large amount of solid was precipitated. The suspension was filtered and dried to give the desired product (25 mg) as white solid. LC-MS (ESI+): m/z 363 (M+H)⁺; ¹H NMR (300 MHz, DMSO-d6) δ 12.61 (s, 1H), 12.48 (s, 1H), 8.88 (s, 1H), 8.68 (d, J=1.8 Hz, 1H), 8.44 (s, 1H), 7.96-8.09 (m, 4H), 7.88 (d, J=1.8 Hz, 1H), 7.58-7.61 (m, 2H), 5.59 (d, J=6.3 Hz, 2H), 1.12 (t, J=3.3 Hz, 2H), 1.01 (t, J=3.3 Hz, 2H).

### Example 2: Preparation of Compound 2

### 1-((3-hydroxy-5-(naphthalen-2-yl)picolinamido)methyl)cyclobutane-1-carboxylic acid

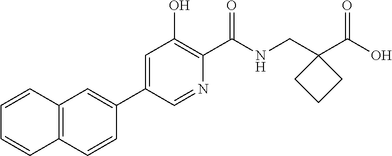 [SMILES: O=C(NCC1(C(=O)O)CCC1)c1ncc(-c2ccc3ccccc3c2)cc1O]

The compound was synthesized according to the procedure described for the preparation of 1-((3-hydroxy-5-(naphthalen-2-yl)picolinamido)methyl)cyclopropane-1-carboxylic acid using ethyl 1-(aminomethyl)cyclobutane-1-carboxylate and 3-hydroxy-5-(naphthalen-2-yl)picolinic acid. LCMS (ESI+): m/z 377.2 (M+H)⁺; 1H-NMR (400 MHz, DMSO-d6) δ 12.51 (s, 2H), 8.97 (s, 1H), 8.65 (d, 1H), 8.41 (s, 1H), 8.06-7.94 (m, 4H), 7.85 (d, 1H), 7.60-7.56 (m, 2H), 3.70 (s, 2H), 2.30 (q, 2H), 2.03-2.00 (m, 2H), 1.97-1.83 (m, 2H).

### Example 3: Preparation of Compound 3

### 1-((5-(3-chlorophenyl)-3-hydroxypicolinamido)methyl)cyclopropane-1-carboxylic acid

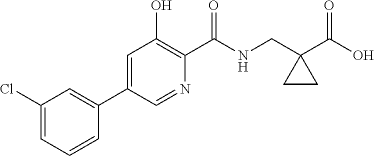 [SMILES: O=C(NCC1(C(=O)O)CC1)c1ncc(-c2cccc(Cl)c2)cc1O]

The compound was synthesized according to the procedure described for the preparation of 1-((3-hydroxy-5-(naphthalen-2-yl)picolinamido)methyl)cyclopropane-1-carboxylic acid using 5-(3-chlorophenyl)-3-hydroxypicolinic acid. LC-MS (ESI+): m/z 347 (M+H)⁺; 1H NMR (300 MHz, CDCl₃) δ 12.21 (s, 1H), 8.59 (t, J=6.6 Hz, 1H), 8.29 (d, J=1.8 Hz, 1H), 7.56 (d, J=0.9 Hz, 1H), 7.40-7.48 (m, 4H), 3.65 (d, J=6.6 Hz, 2H), 1.41-1.44 (m, 2H), 1.11-1.19 (m, 2H).

### Example 4: Preparation of Compound 4

### 1-((5-(3-chlorophenyl)-3-hydroxypicolinamido)methyl)cyclobutane-1-carboxylic acid

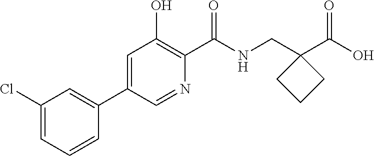 [SMILES: O=C(NCC1(C(=O)O)CCC1)c1ncc(-c2cccc(Cl)c2)cc1O]

The compound was synthesized according to the procedure described for the preparation of 1-((3-hydroxy-5-(naphthalen-2-yl)picolinamido)methyl)cyclopropane-1-carboxylic acid using 5-(3-chlorophenyl)-3-hydroxypicolinic acid and ethyl 1-(aminomethyl)cyclobutane-1-carboxylate. LC-MS (ESI+): m/z 361 (M+H)⁺; 1H NMR (300 MHz, CDCl₃) δ 12.21 (s, 1H), 8.44 (t, J=6.0 Hz, 1H), 8.27 (d, J=1.8 Hz, 1H), 7.55 (d, J=1.2 Hz, 1H), 7.40-7.46 (m, 4H), 3.90 (d, J=6.3 Hz, 2H), 2.51-2.60 (m, 2H), 2.00-2.23 (m, 4H).

### Example 5: Preparation of Compound 5

### 1-((3-hydroxy-5-(1-phenyl-1H-pyrazol-4-yl)picolinamido)methyl)cyclopropane-1-carboxylic acid

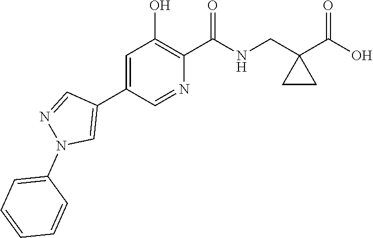 [SMILES: O=C(NCC1(C(=O)O)CC1)c1ncc(-c2cnn(-c3ccccc3)c2)cc1O]

The compound was synthesized according to the procedure described for the preparation of 1-((3-hydroxy-5-(naphthalen-2-yl)picolinamido)methyl)cyclopropane-1-carboxylic acid using 3-hydroxy-5-(1-phenyl-1H-pyrazol-4-yl)picolinic acid. LC-MS (ESI+): m/z 379 (M+H)⁺; 1H NMR (300 MHz, DMSO-d6) δ 12.61 (s, 1H), 12.46 (s, 1H), 9.26 (s, 1H), 8.80 (t, J=6.3 Hz, 1H), 8.60 (d, J=1.8 Hz, 1H), 8.45 (s, 1H), 7.90 (d, J=8.1 Hz, 2H), 7.80 (d, J=1.8 Hz, 1H), 7.56 (t, J=7.8 Hz, 2H), 7.37 (t, J=7.2 Hz, 1H), 3.56 (d, J=6.3 Hz, 2H), 1.14-1.07 (m, 2H), 1.02-0.93 (m, 2H).

### Example 6: Preparation of Compound 6

### (S)-3-(5-(3-chlorophenyl)-3-hydroxypicolinamido)-2-hydroxypropanoic acid

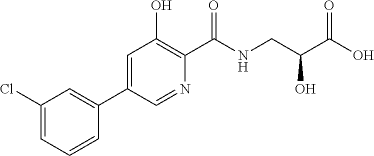 [SMILES: O=C(NC[C@H](O)C(=O)O)c1ncc(-c2cccc(Cl)c2)cc1O]

To a solution of methyl 5-(3-chlorophenyl)-3-hydroxypicolinate (60.00 mg, 0.22 mmol) in MeOH (2 mL) was added in (S)-3-amino-2-hydroxypropanoic acid (0.12 g, 1.10 mmol) and MeONa (36.00 mg, 0.66 mmol) in one portion. The mixture was reacted in microwave reactor at 90° C. for 4 hrs. After the reaction was completed as indicated by LCMS, the reaction was cooled to rt and adjusted pH 3 with diluted HCl solution. A large amount of solid was precipitated. The solid was collected by filtration and purified by preparative HPLC. After lyophillization, 27 mg of desired white solid was obtained. LC-MS (ESI+): m/z 337 (M+H)⁺; ¹H NMR (300 MHz, CD₃OD) δ 8.38 (d, J=1.8 Hz, 1H), 7.82 (s, 1H), 7.62-7.72 (m, 1H), 7.56 (d, J=1.8 Hz, 1H), 7.43-7.52 (m, 2H), 4.22-4.26 (m, 1H), 3.76-3.82 (m, 1H), 3.54-3.70 (m, 1H).

### Example 7: Preparation of Compound 7

### (S)-2-hydroxy-3-(3-hydroxy-5-(naphthalen-2-yl)picolinamido)propanoic acid

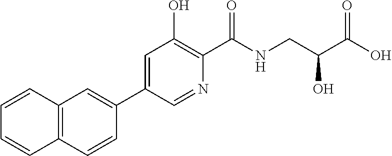 [SMILES: O=C(NC[C@H](O)C(=O)O)c1ncc(-c2ccc3ccccc3c2)cc1O]

The compound was synthesized according to the procedure described for the preparation of (S)-3-(5-(3-chlorophenyl)-3-hydroxypicolinamido)-2-hydroxypropanoic acid using 3-hydroxy-5-(naphthalen-2-yl)picolinic acid. LC-MS (ESI+): m/z 353 (M+H)⁺; ¹H NMR (300 MHz, DMSO-d6) δ 12.52 (s, 1H), 8.95 (s, 1H), 8.67 (s, 1H), 8.43 (s, 1H), 8.07 (d, J=8.7 Hz, 2H), 7.98 (s, 2H), 7.87 (s, 1H), 7.60 (s, 2H), 4.27 (s, 1H), 3.58-3.63 (m, 2H).

### Example 8: Preparation of Compound 8

### (R)-2-hydroxy-3-(3-hydroxy-5-(naphthalen-2-yl)picolinamido)propanoic acid

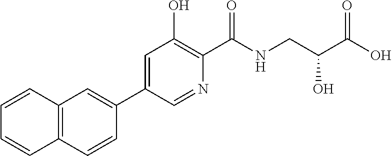 [SMILES: O=C(NC[C@@H](O)C(=O)O)c1ncc(-c2ccc3ccccc3c2)cc1O]

The compound was synthesized according to the procedure described for the preparation of (S)-3-(5-(3-chlorophenyl)-3-hydroxypicolinamido)-2-hydroxypropanoic acid using 3-hydroxy-5-(naphthalen-2-yl)picolinic acid. LC-MS (ESI+): m/z 353 (M+H)⁺; ¹H NMR (300 MHz, DMSO-d6) δ 12.73 (d, J=3.6 Hz, 1H), 12.53 (s, 1H), 8.96 (s, 1H), 8.67 (s, 1H), 8.44 (s, 1H), 8.03-8.09 (m, 2H), 7.97 (d, J=9.6 Hz, 2H), 7.87 (s, 1H), 7.60 (s, 2H), 5.70 (s, 1H), 4.27 (s, 1H), 3.53-3.69 (m, 2H).

### Example 9: Preparation of Compound 9

### (R)-3-(5-(3-chlorophenyl)-3-hydroxypicolinamido)-2-hydroxypropanoic acid

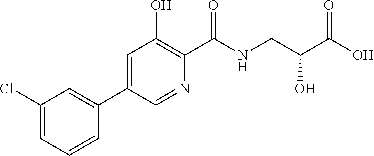 [SMILES: O=C(NC[C@@H](O)C(=O)O)c1ncc(-c2cccc(Cl)c2)cc1O]

The compound was synthesized according to the procedure described for the preparation of (S)-3-(5-(3-chlorophenyl)-3-hydroxypicolinamido)-2-hydroxypropanoic acid using methyl 5-(3-chlorophenyl)-3-hydroxypicolinate. LC-MS (ESI+): m/z 337 (M+H)⁺; ¹H NMR (300 MHz, CD₃OD) δ 8.40 (d, J=1.8 Hz, 1H), 7.73 (s, 1H), 7.67-7.60 (m, 1H), 7.58 (d, J=1.8 Hz, 1H), 7.54-7.43 (m, 2H), 4.38 (dd, J=4.5 Hz, J=6.9 Hz, 1H), 3.82 (dd, J=4.5 Hz, J=13.7 Hz, 1H), 3.67-3.73 (m, 1H).

### Example 10: Preparation of Compound 10

### (S)-2-hydroxy-3-(3-hydroxy-5-(1-phenyl-1H-pyrazol-4-yl)picolinamido)propanoic acid

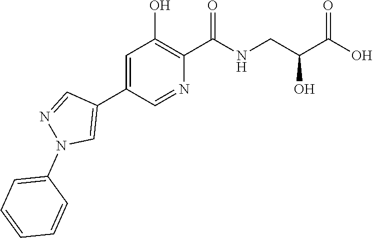 [SMILES: O=C(NC[C@H](O)C(=O)O)c1ncc(-c2cnn(-c3ccccc3)c2)cc1O]

The compound was synthesized according to the procedure described for the preparation of (S)-3-(5-(3-chlorophenyl)-3-hydroxypicolinamido)-2-hydroxypropanoic acid using methyl 3-hydroxy-5-(1-phenyl-1H-pyrazol-4-yl)picolinate. LC-MS (ESI+): m/z 369 (M+H)⁺; ¹H NMR (300 MHz, DMSO-d₆) δ 12.50 (s, 1H), 9.26 (s, 1H), 8.87 (s, 1H), 8.59 (s, 1H), 8.45 (s, 1H), 7.89 (d, J=4.8 Hz, 2H), 7.79 (s, 1H), 7.56 (t, J=7.5 Hz, 2H), 7.37 (t, J=7.5 Hz, 1H), 4.24 (t, J=5.7 Hz, 1H), 3.53-3.64 (m, 2H).

### Example 11: Preparation of Compound 11

### (R)-2-hydroxy-3-(3-hydroxy-5-(1-phenyl-1H-pyrazol-4-yl)picolinamido)propanoic

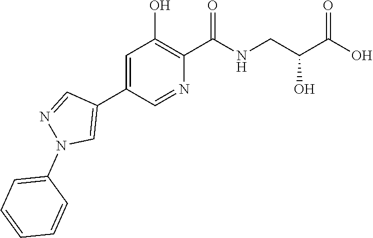 [SMILES: O=C(NC[C@@H](O)C(=O)O)c1ncc(-c2cnn(-c3ccccc3)c2)cc1O]

The compound was synthesized according to the procedure described for the preparation of (S)-3-(5-(3-chlorophenyl)-3-hydroxypicolinamido)-2-hydroxypropanoic acid using methyl 3-hydroxy-5-(1-phenyl-1H-pyrazol-4-yl)picolinate. LC-MS (ESI+): m/z 369 (M+H)⁺; ¹H NMR (300 MHz, DMSO-d₆) δ 12.54 (brs, 1H), 9.26 (s, 1H), 8.91 (d, J=6.0 Hz, 1H), 8.59 (d, J=1.2 Hz, 1H), 8.45 (s, 1H), 7.90 (d, J=7.8 Hz, 2H), 7.78 (d, J=1.5 Hz, 1H), 7.56 (t, J=7.5 Hz, 2H), 7.37 (t, J=7.5 Hz, 1H), 4.15 (t, J=5.7 Hz, 1H), 3.56 (t, J=5.7 Hz, 2H).

### Example 12: Preparation of Compound 12

### 1-((3-hydroxy-5-(1-phenyl-1H-pyrazol-4-yl)picolinamido)methyl)cyclobutane-1-carboxylic acid

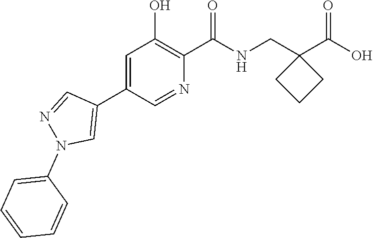 [SMILES: O=C(NCC1(C(=O)O)CCC1)c1ncc(-c2cnn(-c3ccccc3)c2)cc1O]

The compound was synthesized according to the procedure described for the preparation of 1-((3-hydroxy-5-(naphthalen-2-yl)picolinamido)methyl)cyclopropane-1-carboxylic acid using ethyl 1-(aminomethyl)cyclobutane-1-carboxylate and 3-hydroxy-5-(1-phenyl-1H-pyrazol-4-yl)picolinic acid. LC-MS (ESI+): m/z 393 (M+H)⁺; ¹H NMR (300 MHz, CD₃OD) δ 8.84 (s, 1H), 8.47 (d, J=1.5 Hz, 1H), 8.22 (s, 1H), 7.83 (d, J=7.8 Hz, 2H), 7.62 (d, J=1.8 Hz, 1H), 7.52 (t, J=7.8 Hz, 2H), 7.37 (t, J=7.2 Hz, 1H), 3.80 (s, 2H), 2.41-2.47 (m, 2H), 1.97-2.14 (m, 4H).

### Example 13: Preparation of Compound 13

### 1-((3-hydroxy-5-(1-phenyl-1H-pyrazol-4-yl)picolinamido)methyl)cyclohexane-1-carboxylic acid

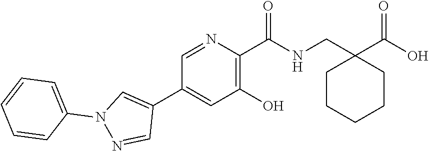 [SMILES: O=C(NCC1(C(=O)O)CCCCC1)c1ncc(-c2cnn(-c3ccccc3)c2)cc1O]

The compound was synthesized according to the procedure described for the preparation of 1-((3-hydroxy-5-(naphthalen-2-yl)picolinamido)methyl)cyclopropane-1-carboxylic acid using ethyl 1-(aminomethyl)cyclohexane-1-carboxylate and 3-hydroxy-5-(1-phenyl-1H-pyrazol-4-yl)picolinic acid. LC-MS (ESI+): m/z 421 (M+H)⁺; ¹H NMR (300 MHz, CD₃OD) δ 8.82 (s, 1H), 8.48 (d, J=1.8 Hz, 1H), 8.21 (d, J=5.1 Hz, 1H), 7.84 (d, J=7.5 Hz, 2H), 7.62 (d, J=1.5 Hz, 1H), 7.54 (t, J=7.8 Hz, 2H), 7.38 (t, J=7.5 Hz, 1H), 3.60 (s, 2H), 2.06-2.11 (m, 2H), 1.61-1.66 (m, 2H), 1.39-1.59 (m, 6H).

### Example 14: Preparation of Compound 14

### 4-((3-hydroxy-5-(1-phenyl-1H-pyrazol-4-yl)picolinamido)methyl)tetrahydro-2H-pyran-4-carboxylic acid

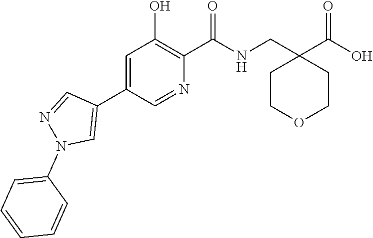 [SMILES: O=C(NCC1(C(=O)O)CCOCC1)c1ncc(-c2cnn(-c3ccccc3)c2)cc1O]

The compound was synthesized according to the procedure described for the preparation of 1-((3-hydroxy-5-(naphthalen-2-yl)picolinamido)methyl)cyclopropane-1-carboxylic acid using ethyl 4-(aminomethyl)tetrahydro-2H-pyran-4-carboxylate and 3-hydroxy-5-(1-phenyl-1H-pyrazol-4-yl)picolinic acid. LC-MS (ESI+): m/z 423 (M+H)⁺; ¹H NMR (300 MHz, CD₃OD) δ 8.73 (s, 1H), 8.38 (d, J=1.8 Hz, 1H), 8.10 (d, J=6.0 Hz, 1H), 7.73 (d, J=7.8 Hz, 2H), 7.52 (d, J=1.8 Hz, 1H), 7.43 (t, J=7.5 Hz, 2H), 7.27 (t, J=7.5 Hz, 1H), 3.73-3.78 (m, 2H), 3.55 (s, 2H), 3.44-3.52 (m, 2H), 1.99-2.03 (m, 2H), 1.48-1.57 (m, 2H).

### Example 15: Preparation of Compound 15

### 2-(1-(3-hydroxy-5-(1-phenyl-1H-pyrazol-4-yl)picolinamido)cyclopropyl)acetic acid

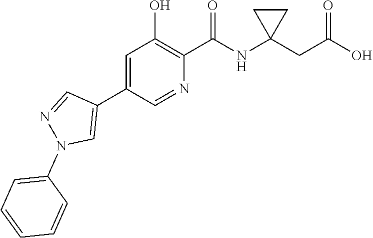 [SMILES: O=C(O)CC1(NC(=O)c2ncc(-c3cnn(-c4ccccc4)c3)cc2O)CC1]

The compound was synthesized according to the procedure described for the preparation of 1-((3-hydroxy-5-(naphthalen-2-yl)picolinamido)methyl)cyclopropane-1-carboxylic acid using ethyl 2-(1-aminocyclopropyl)acetate and 3-hydroxy-5-(1-phenyl-1H-pyrazol-4-yl)picolinic acid. LC-MS (ESI+): m/z 379 (M+H)⁺; ¹H NMR (300 MHz, CD₃OD) δ 8.72 (s, 1H), 8.35 (d, J=1.5 Hz, 1H), 8.10 (s, 1H), 7.73 (d, J=7.8 Hz, 2H), 7.50 (d, J=1.5 Hz, 1H), 7.42 (t, J=7.5 Hz, 2H), 7.24-7.29 (m, 1H), 2.62 (s, 2H), 0.90-0.94 (m, 2H), 0.78-0.85 (m, 2H).

### Example 16: Preparation of Compound 16

### 2-(1-(3-hydroxy-5-(1-phenyl-1H-pyrazol-4-yl)picolinamido)cyclobutyl)acetic acid

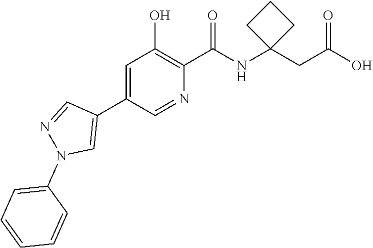 [SMILES: O=C(O)CC1(NC(=O)c2ncc(-c3cnn(-c4ccccc4)c3)cc2O)CCC1]

The compound was synthesized according to the procedure described for the preparation of 1-((3-hydroxy-5-(naphthalen-2-yl)picolinamido)methyl)cyclopropane-1-carboxylic acid using ethyl 2-(1-aminocyclobutyl)acetate and 3-hydroxy-5-(1-phenyl-1H-pyrazol-4-yl)picolinic acid. LC-MS (ESI+): m/z 393 (M+H)⁺; ¹H NMR (300 MHz, CD₃OD) δ 8.73 (s, 1H), 8.36 (d, J=1.5 Hz, 1H), 8.11 (s, 1H), 7.73 (d, J=7.8 Hz, 2H), 7.52 (d, J=1.5 Hz, 1H), 7.43 (t, J=7.5 Hz, 2H), 7.27 (t, J=7.5 Hz, 1H), 2.94 (s, 2H), 2.31-2.48 (m, 2H), 2.22-2.29 (m, 2H), 1.85-1.93 (m, 2H).

### Example 17: Preparation of Compound 17

### 1-((3-hydroxy-5-(1-phenyl-1H-pyrazol-4-yl)picolinamido)methyl)cyclopentane-1-carboxylic acid

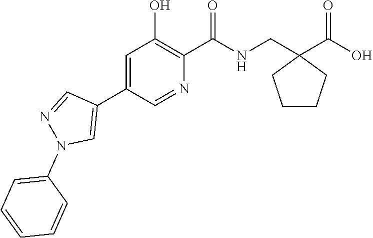 [SMILES: O=C(NCC1(C(=O)O)CCCC1)c1ncc(-c2cnn(-c3ccccc3)c2)cc1O]

The compound was synthesized according to the procedure described for the preparation of 1-((3-hydroxy-5-(naphthalen-2-yl)picolinamido)methyl)cyclopropane-1-carboxylic acid using ethyl 1-(aminomethyl)cyclopentane-1-carboxylate and 3-hydroxy-5-(1-phenyl-1H-pyrazol-4-yl)picolinic acid. LC-MS (ESI+): m/z 407 (M+H)⁺; 1H NMR (300 MHz, CD₃OD) δ 8.72 (s, 1H), 8.38 (d, J=8.1 Hz, 1H), 8.10 (d, J=6.0 Hz, 1H), 7.73 (d, J=7.5 Hz, 2H), 7.52 (d, J=1.8 Hz, 1H), 7.40-7.45 (m, 2H), 7.27 (t, J=7.8 Hz, 1H), 3.50 (s, 2H), 1.90-2.00 (m, 2H), 1.58-1.71 (m, 6H).

### Example 18: In Vitro Assays Demonstrate PHD1 Selectivity Over PHD2 and/or PHD3

Enzymatic half maximal inhibitory concentration (IC₅₀) values were determined on selected compounds of the invention. The compounds have an IC₅₀ value of less than 50 μM against PHD1 and showed an increase in selectivity for PHD1 over PHD2.

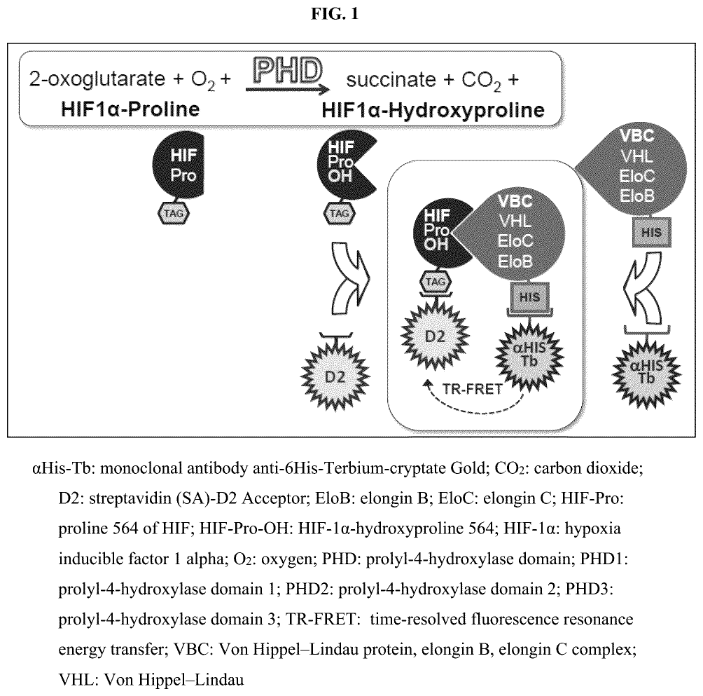

Time-resolved fluorescence resonance energy transfer (TR-FRET) assay was utilized to determine the enzymatic half maximal inhibitory concentration (IC₅₀) value of PHD inhibitors against the full-length human prolyl-4-hydroxylase domain (PHD) enzymes, PHD1 and PHD2. The TR-FRET assay was developed based on the specific binding of hydroxylated HIF-1α peptide with the complex formed by VHL, EloB and EloC (VBC), to generate a fluorescent signal. Terbium (Tb)-Donor (monoclonal antibody anti-6His-Tb-cryptate Gold) and D2-acceptor (streptavidin [SA]-D2) of TR-FRET are linked to the VBC complex and to HIF-1a peptide, respectively. The VBC complex binds specifically to the HIF-1a peptide when it is hydroxylated, allowing energy transfer from TR-FRET donor to acceptor (FIG. 1).

### Materials and Methods

All chemicals and materials unless otherwise noted were of standard laboratory grade and were purchased from Sigma-Aldrich (St. Louis, MO, USA).

### Reagents

TR-FRET Reagents. Monoclonal antibody anti-6His-Tb-cryptate Gold (catalog #61HI2TLA) and streptavidin (SA)-D2 (catalog #610SADLA) were purchased from CisBio International (Bedford, MA, USA).

N-terminus biotinylated HIF-1α C₃₅ synthetic peptide representing amino acids 547 to 581 and including the proline 564 PHD2 hydroxylation site was purchased from California Peptide Research (Salt Lake City, UT, USA).

### Recombinant Proteins

VBC complex. His-tagged recombinant VHL protein, EloB, EloC complex (His-VBC) was supplied by Axxam (Milan, Italy). Recombinant human VHL (National Center for Biotechnology Information [NCBI] accession number NP_00542.1) contained a His tag at the C-terminus of amino acids 55 to 213 and is referred to as VHL-His. VHL-His was co-expressed in E. coli with full-length human EloB (NCBI accession number Q15370.1) and full-length human EloC (NCBI accession number Q15369.1) and purified by affinity chromatography on a nickel-nitrilotriacetic acid (Ni-NTA) column as the His-VBC complex. Purity (˜80%) was assessed by sodium dodecyl sulfate polyacrylamide gel electrophoresis (SDS-PAGE).

PHD1. Recombinant human PHD1 protein (catalog #81064, Lot #24717001) was purchased from Active Motif (Carlsbad, CA, USA). PHD1 was expressed in a baculovirus expression system as the full-length protein (NCBI accession number NP_542770.2) with an N-terminal FLAG tag (molecular weight 44.9 kDa). Purity (>90%) was assessed by SDS-PAGE.

PHD2. The full-length human PHD2 enzyme was produced with a baculovirus infected insect cell (BIIC) expression system by Beryllium (Bedford, MA, USA). The PHD2 construct contained amino acids 1 to 426 of PHD2 (UniProt Knowledgebase[UniProtKB]/Swiss-Prot accession number Q9GZT9.1), and a His tag and a Tobacco Etch Virus (TEV) protease cleavage site at the N-terminus. The construct was expressed in Sf9 insect cells, purified by Ni-NTA column and digested with TEV protease to remove the His tag. The purity of final cleaved protein was assessed by SDS-PAGE and was found to be >94% pure.

PHD Inhibitors. Small molecule PHD inhibitors were synthesized and their identities were confirmed as described herein.

TR-FRET Assay Procedure. PHD inhibitor compound was preincubated with PHD enzyme in a 10 μL reaction volume in white 384-well Optiplate microplates (catalog #6007290, Perkin Elmer, Waltham, MA, USA). For this, 5 μL of PHD inhibitor compound was serially diluted with dilution buffer (50 mM HEPES [4-(2-hydroxyethyl)-1-piperazineethanesulfonic acid] pH 7.5, 50 mM sodium chloride [NaCl], 0.01% Tween-20, 0.01% purified bovine serum albumin [BSA]) and mixed with 5 μL PHD enzyme mix prepared as a 4× concentrate in the dilution buffer containing PHD enzyme (60 nM PHD1, 20 nM PHD2, 140 nM PHD3), 40 μM ferrous ammonium sulfate (FAS), 4 mM sodium (Na) ascorbate. The plates were incubated for 30 minutes at room temperature without rotation.

Five microliters of the VBC/anti-6His-Tb-cryptate Gold mix prepared as a 4× concentrate in dilution buffer containing 20 nM His-VBC, 1.32 nM monoclonal antibody anti-6His-Tb-cryptate Gold was then added. This step was followed immediately by the addition of 5 L of the HIF-1a C35 substrate mix prepared as a 4× concentrate in the dilution buffer containing 120 nM biotin-labeled HIF-1a C35, 132 nM SA-D2, 4 μM 2-oxoglutarate (2-OG) to reach a final reaction volume of 20 μL.

The final assay reaction contained 50 mM HEPES, pH 7.5, 50 mM NaCl, 1 μM 2-OG, M FAS, 1 mM Na ascorbate, 0.01% Tween-20, 0.01% purified BSA, 30 nM biotin-labeled HIF-1a C35, 5 nM His-VBC, 0.33 nM monoclonal antibody anti-6His-Tb-cryptate Gold, 33 nM SA-D2 and PHD enzyme (15 nM PHD1, 5 nM PHD2, or 35 nM PHD3) with the diluted compound.

For the measurement of the IC₅₀ of PHD inhibitor compound, reactions were incubated for 10 minutes at room temperature and then read on a Perkin Elmer EnVision (Waltham, MA, USA) at an excitation wavelength of 340 nm and at emission wavelengths of 615 nm and 665 nm. The data represent the quotient of the signal intensity at 665 nm and 615 nm, automatically calculated by Envision Manager software (Perkin Elmer, Waltham, MA, USA). The IC₅₀ values (mean, standard deviation, standard error of the mean, geometric mean and 95% confidence interval) were determined using a four-parameter curve-fit using GraphPad Prism 7.0 (GraphPad, La Jolla, CA, USA) and represent the compound concentration plotted against the calculated ratio of 665 nm and 615 nm. TR-FRET assays were performed in triplicate at each concentration of compound and the assays were repeated independently three times.

Selectivity of compounds for PHD1 over PHD2 was determined by taking ratios of Kis in the respective assays.

Kis were calculated from IC₅₀s based on the Cheng Prussoff equation:

Ki=IC50/(1+[2−OG]/Km)

The final concentration of 2-OG in both the PHD1 and PHD2 assays is 1 uM. The Km of 2-OG for PHD1 was determined to be 12.7 nM, while the Km of 2-OG for PHD2 was determined to be 22.6 nM.

### Exemplary Compounds

| Cmpd. | | Selectivity of PHD1 over |
|---|---|---|
| No. | Structure | PHD2 |
| 1 | 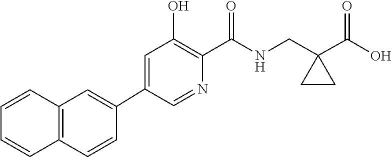 [SMILES: O=C(NCC1(C(=O)O)CC1)c1ncc(-c2ccc3ccccc3c2)cc1O] | ~219 fold |
| 2 | 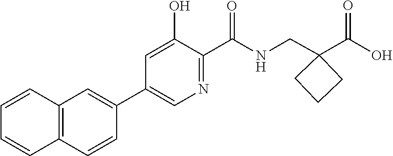 [SMILES: O=C(NCC1(C(=O)O)CCC1)c1ncc(-c2ccc3ccccc3c2)cc1O] | ~1258 fold |
| 3 | 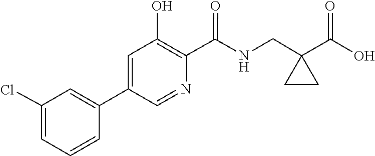 [SMILES: O=C(NCC1(C(=O)O)CC1)c1ncc(-c2cccc(Cl)c2)cc1O] | ~338 fold |
| 4 | 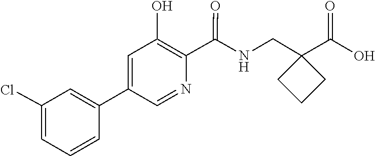 [SMILES: O=C(NCC1(C(=O)O)CCC1)c1ncc(-c2cccc(Cl)c2)cc1O] | ~6 fold |
| 5 | 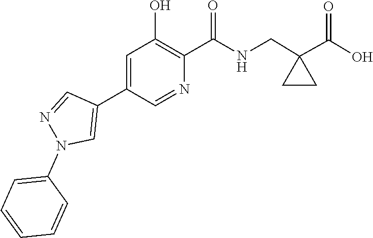 | ~61 fold |
| 6 | 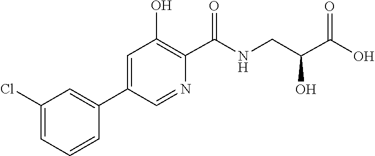 [SMILES: O=C(NC[C@H](O)C(=O)O)c1ncc(-c2cccc(Cl)c2)cc1O] | ~11 fold |
| 7 | 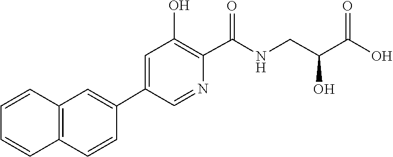 [SMILES: O=C(NC[C@H](O)C(=O)O)c1ncc(-c2ccc3ccccc3c2)cc1O] | ~16 fold |
| 8 | 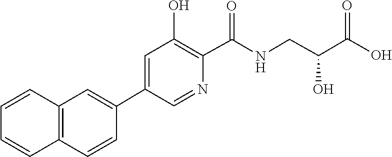 [SMILES: O=C(NC[C@@H](O)C(=O)O)c1ncc(-c2ccc3ccccc3c2)cc1O] | ~100 fold |
| 9 | 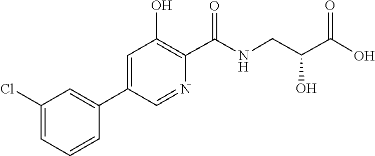 [SMILES: O=C(NC[C@@H](O)C(=O)O)c1ncc(-c2cccc(Cl)c2)cc1O] | ~13 fold |
| 10 | 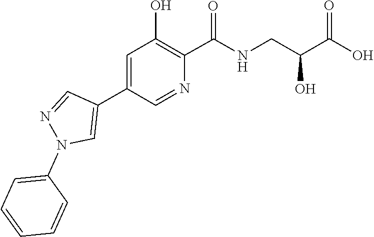 | ~29 fold |
| 11 | 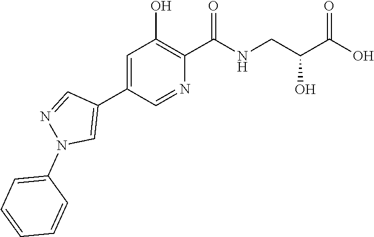 | ~42 fold |
| 12 | 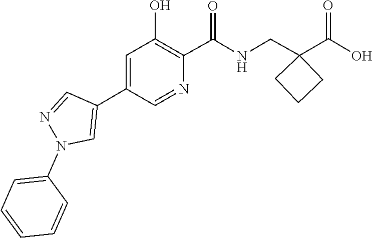 [SMILES: O=C(NCC1(C(=O)O)CCC1)c1ncc(-c2cnn(-c3ccccc3)c2)cc1O] | ~36 fold |
| 13 | 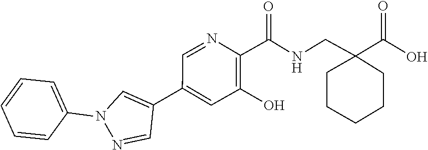 [SMILES: O=C(NCC1(C(=O)O)CCCCC1)c1ncc(-c2cnn(-c3ccccc3)c2)cc1O] | ~2 fold |
| 14 | 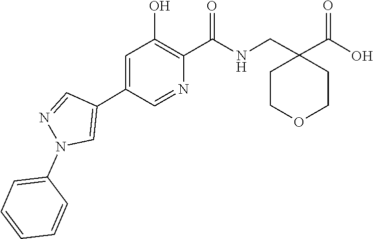 [SMILES: O=C(NCC1(C(=O)O)CCOCC1)c1ncc(-c2cnn(-c3ccccc3)c2)cc1O] | ~51 fold |
| 15 | 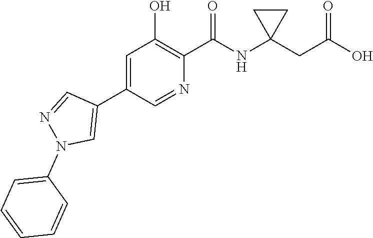 | ~10 fold |
| 16 | 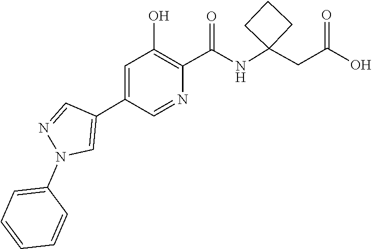 | ~2 fold |
| 17 | 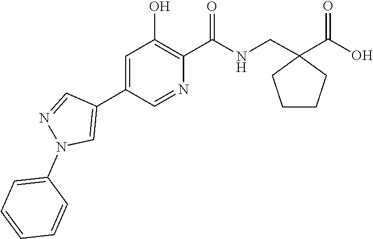 | ~8 fold |

From the foregoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

What is claimed is:

1. A compound of Formula (I): 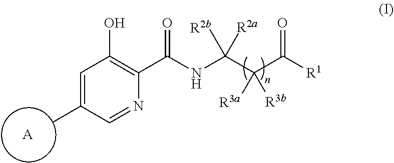
or a pharmaceutically acceptable salt thereof, wherein:
A is aryl or heteroaryl, optionally substituted with aryl, heteroaryl, halo, C₁-C₄ alkyl, alkoxy, aryloxy, heteroaryloxy, amino, arylamino, heteroarylamino, amide, cyano, nitro, sulfonamide;
R¹ is OH or optionally substituted ester;
R^{2a}, R^{2b} R^{3a}, and R^{3b} are each independently H, OH, C₁-C₄ alkyl, provided that at least one of R^{2a}, R^{2b} R^{3a}, or R^{3b} is OH; or
R^{2a} and R^{2b} are independently H, OH, or C₁-C₄ alkyl; or R^{2a} and R^{2b} together with the carbon to which they are attached form a 3-6 membered cycloalkyl, which is optionally substituted with C₁-C₃ alkyl or substituted C₁-C₄ alkyl; or R^{2a} and R^{2b} together with the carbon to which they are attached form a 3-6 membered heterocycloalkyl, which is optionally substituted with C₁-C₃ alkyl; and R^{3a} and R^{3b} are independently H, OH, or C₁-C₄ alkyl; or R^{3a} and R^{3b} together with the carbon to which they are attached form a 3-6 membered cycloalkyl, which is optionally substituted with C₁-C₃ alkyl or substituted C₁-C₄ alkyl; or R^{3a} and R^{3b} together with the carbon to which they are attached from a 3-6 membered heterocycloalkyl, which is optionally substituted with C₁-C₃ alkyl;
n is 1 or 2; and
wherein at least one of R^{2a}, R^{2b}, R^{3a} and R^{3b} is not H.

2. The compound of claim 1, wherein A is 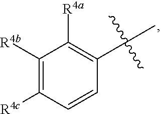
wherein
R^{4a}, R^{4b}, and R^{4c} are independently H, halo, aryl, heteroaryl, CH₂OR¹², OR¹², NHR¹² or CH₂R¹³;
R¹² is H, aryl optionally substituted with R¹⁴, or C₁-C₂ alkyl optionally substituted with R¹⁵;
R¹³ is a heterocycloalkyl;
R¹⁴ is H or halo; and
R¹⁵ is cycloalkyl or aryl optionally substituted with halo.

3. The compound of claim 1, wherein A is 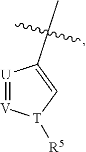
wherein
U, V, and T are independently CH, or N;
R⁵ is C₁-C₄ alkyl optionally substituted with R¹⁶; aryl optionally substituted with H, halo, or CF₃; or heteroaryl optionally substituted with CO₂R¹⁷;
R¹⁶ is H, aryl optionally substituted with halo, or heterocycloalkyl; and
R¹⁷ is t-butyl.

4. The compound of claim 3, wherein A is 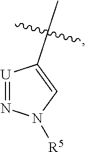
wherein
U is CH or N;
R⁵ is C₁-C₄ alkyl optionally substituted with R¹⁶; aryl optionally substituted with H, halo, or CF₃; heterocycloalkyl optionally substituted with CO₂R¹⁷; or heteroaryl optionally substituted with CO₂R¹⁷;
R¹⁶ is H, aryl optionally substituted with halo, or heterocyclic; and
R¹⁷ is t-butyl.

5. The compound of claim 1, wherein A is 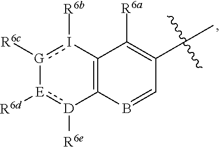
wherein
B, D, E, G, and I are independently C, CH, or N;
R^{6a}, R^{6b}, R^{6c}, and R^{6d} are independently H, C₁-C₃ alkyl, halo, OR¹⁸, or NHR¹⁹, R^{6e} is H or ═O;
R¹⁸ is H, aryl optionally substituted with halo, or C₁-C₃ alkyl optionally substituted with R²⁰;
R¹⁹ is SO₂CH₃;
R²⁰ is aryl optionally substituted with halo; and
----- is an optional bond.

6. The compound of claim 5, wherein A is 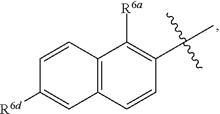
wherein
R^{6a} is H or methyl;
R^{6d} is H, OR¹⁸, or NHR¹⁹,
R¹⁸ is H, aryl optionally substituted with halo, or C₁-C₃ alkyl optionally substituted with R²⁰;
R¹⁹ is SO₂CH₃; and
R²⁰ is aryl optionally substituted with halo.

7. The compound of claim 5, wherein A is 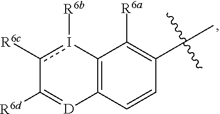
wherein
D is CH, CR^{6e} or N;
I is N, C, or CH;
R^{6a} is H or halo;
R^{6b} is H or C₁-C₃ alkyl;
R^{6c}, is H, ═O or C₁-C₃ alkyl;
R^{6d} is H or C₁-C₃ alkyl;
R^{6e} is H or ═O; and
----- is an optional bond.

8. The compound of claim 5, wherein A is 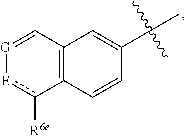
wherein
G is CH or N;
E is CH, CH₂, N, or NH;
R^{6e} is H or ═O; and
---- is an optional bond.

9. The compound of claim 1, wherein A is 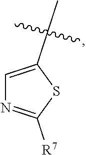
wherein
R⁷ is H, C₁-C₃ alkyl, or phenyl.

10. The compound of claim 1, wherein A is 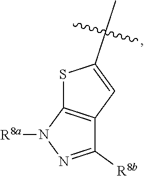
wherein
R^{8a} and R^{8b} are independently H or C₁-C₃ alkyl.

11. The compound of claim 1, wherein A is 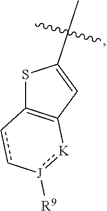
wherein
J is C, CH, or N;
K is CH, CH₂, N, or NH;
R⁹ is H, halo, C₁-C₄ alkyl, or CO₂R²¹;
R²¹ is t-butyl; and
------ is an optional bond.

12. The compound of claim 11, wherein A is 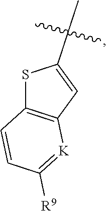
wherein
K is CH or N; and
R⁹ is H, halo, or C₁-C₄ alkyl.

13. The compound of claim 11, wherein A is 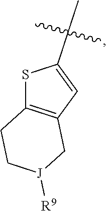
wherein
J is CH or N;
R⁹ is H or CO₂R²¹; and
R²¹ is t-butyl.

14. The compound of claim 1, wherein A is 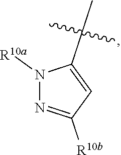
wherein
R^{10a} is H or C₁-C₃ alkyl; and
R^{10b} is H or thiazole.

15. The compound of claim 1, wherein A is 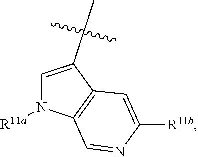
wherein
R^{11a} and R^{11b} are independently H, C₁-C₃ alkyl, or C₁-C₃ alkoxy.

16. The compound of claim 1, wherein A is

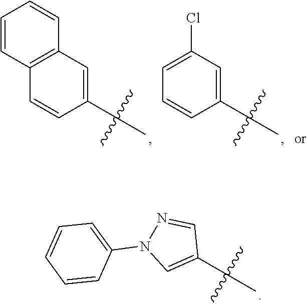 [SMILES: *c1ccc2ccccc2c1 ; *c1cccc(Cl)c1 ; *c1cnn(-c2ccccc2)c1]

17. A compound of Formula (II): 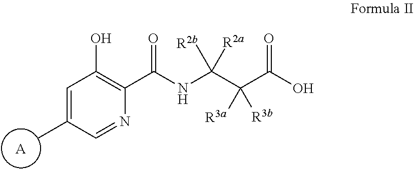
or a pharmaceutically acceptable salt thereof, wherein:
A is aryl or heteroaryl, optionally substituted with aryl, halo, C₁-C₄ alkyl, alkoxy, aryloxy, heteroaryloxy, amino, arylamino, heteroarylamino, amide, cyano, nitro, sulfonamide;
R^{2a}, R^{2b} R^{3a}, and R^{3b} are each independently H, OH, C₁-C₄ alkyl, provided that at least one of R^{2a}, R^{2b} R^{3a}, or R^{3b} is OH; or
R^{2a} and R^{2b} are independently H, OH, or C₁-C₄ alkyl; or R^{2a} and R^{2b} together with the carbon to which they are attached form a 3-6 membered cycloalkyl, optionally substituted by substituted C₁-C₄ alkyl; or R^{2a} and R^{2b} together with the carbon to which they are attached form a 3-6 membered heterocycloalkyl; and R^{3a} and R^{3b} are independently H, OH, or C₁-C₄ alkyl; or R^{3a} and R^{3b} together with the carbon to which they are attached form a 3-6 membered cycloalkyl, optionally substituted by substituted C₁-C₄ alkyl; or R^{3a} and R^{3b} together with the carbon to which they are attached form a 3-6 membered heterocycloalkyl.

18. The compound of claim 17, wherein A is 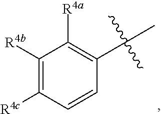
wherein
R^{4a}, R^{4b}, and R^{4c} are independently H, halo, aryl, heteroaryl, CH₂OR¹², OR¹², NHR¹² or CH₂R¹³;
R¹² is H, aryl optionally substituted with R¹⁴, or C₁-C₂ alkyl optionally substituted with R¹⁵;
R¹³ is heterocycloalkyl;
R¹⁴ is H or halo; and
R¹⁵ is cycloalkyl or aryl optionally substituted with halo.

19. The compound of claim 18, wherein R¹³ is pyrrolidine.

20. The compound of claim 17, wherein A is 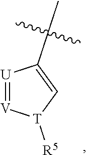
wherein
U, V, and T are independently CH, or N;
R⁵ is C₁-C₄ alkyl optionally substituted with R¹⁶; aryl optionally substituted with H, halo, CF₃; or heteroaryl optionally substituted with CO₂R¹⁷;
R¹⁶ is H, aryl optionally substituted with halo, or heterocycloalkyl; and
R¹⁷ is t-butyl.

21. The compound of claim 20, wherein A is 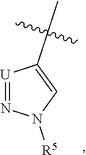
wherein
U is CH or N;
R⁵ is C₁-C₄ alkyl optionally substituted with R¹⁶; aryl optionally substituted with H, halo, or CF₃; heterocycloalkyl optionally substituted with CO2R¹⁷; or heteroaryl optionally substituted with CO₂R¹⁷;
R¹⁶ is H, aryl optionally substituted with halo, or heterocyclic; and
R¹⁷ is t-butyl.

22. The compound of claim 17, wherein A is 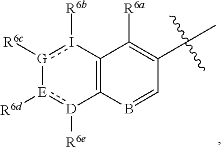
wherein
B, D, E, G, and I are independently C, CH, or N;
R^{6a}, R^{6b}, R^{6c}, and R^{6d} are independently H, C₁-C₃ alkyl, halo, OR¹⁸, or NHR¹⁹, R^{6e} is H or ═O;
R¹⁸ is H, aryl optionally substituted with halo, or C₁-C₃ alkyl optionally substituted with R²⁰;
R¹⁹ is SO₂CH₃;
R²⁰ is aryl optionally substituted with halo; and
wherein------- is an optional bond.

23. The compound of claim 22, wherein A is 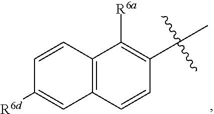
wherein
R^{6a} is H or methyl;
R^{6d} is H, OR¹⁸, or NHR¹⁹,
R¹⁸ is H, aryl optionally substituted with halo, or C₁-C₃ alkyl optionally substituted with R²⁰;
R¹⁹ is SO₂CH₃; and
R²⁰ is aryl optionally substituted with halo.

24. The compound of claim 22, wherein A is 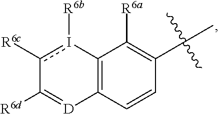
wherein
D is CH, CR^{6e} or N;
I is C, CH, or N;
R^{6a} is H or halo;
R^{6b} is H or C₁-C₃ alkyl;
R^{6c} is H, ═O or C₁-C₃ alkyl;
R^{6d} is H or C₁-C₃ alkyl;
R^{6e} is H or ═O; and
----- is an optional bond.

25. The compound of claim 22, wherein A is 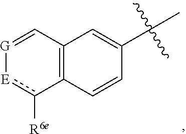
wherein
G is CH, or N;
E is CH, CH₂, N, or NH;
R^{6e} is H or ═O; and
---- is an optional bond.

26. The compound of claim 17, wherein A is 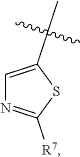
wherein
R⁷ is H, C₁-C₃ alkyl, or phenyl.

27. The compound of claim 17, wherein A is 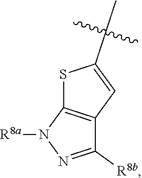
wherein
R^{8a} and R^{8b} are independently H or C₁-C₃ alkyl.

28. The compound of claim 17, wherein A is 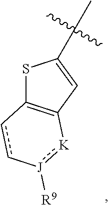
wherein
J is C, CH, or N;
K is CH, CH₂, N, or NH;
R⁹ is H, halo, C₁-C₄ alkyl, or CO₂R²¹;
R²¹ is t-butyl; and
------ is an optional bond.

29. The compound of claim 28, wherein A is 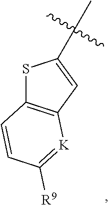
wherein
K is CH or N; and
R⁹ is H, halo, or C₁-C₄ alkyl.

30. The compound of claim 28, wherein A is 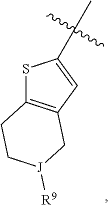
wherein
J is CH or N;
R⁹ is H or CO₂R²¹; and
R²¹ is t-butyl.

31. The compound of claim 17, wherein A is 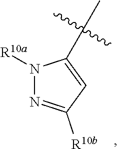
wherein
R^{10a} is H or C₁-C₃ alkyl; and
R^{10b} is H or thiazole.

32. The compound of claim 17, wherein A is 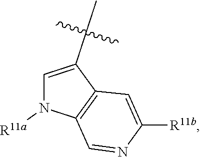
wherein
R^{11a} and R^{11b} are independently H, C₁-C₃ alkyl, or C₁-C₃ alkoxy.

33. The compound of claim 17, which is selected from the group consisting of any one of compounds 1-17, or a pharmaceutically acceptable salt thereof:

| Cmpd. | |
|---|---|
| No. | Structure |
| 1 | 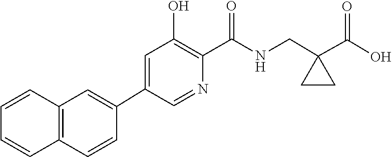 [SMILES: O=C(NCC1(C(=O)O)CC1)c1ncc(-c2ccc3ccccc3c2)cc1O] |
| 2 | 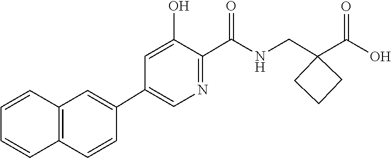 [SMILES: O=C(NCC1(C(=O)O)CCC1)c1ncc(-c2ccc3ccccc3c2)cc1O] |
| 3 | 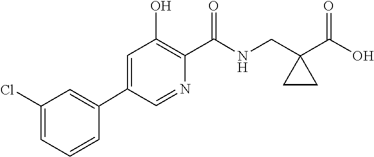 [SMILES: O=C(NCC1(C(=O)O)CC1)c1ncc(-c2cccc(Cl)c2)cc1O] |
| 4 | 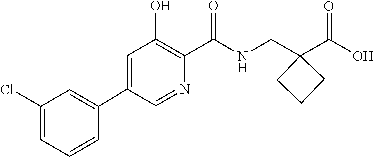 [SMILES: O=C(NCC1(C(=O)O)CCC1)c1ncc(-c2cccc(Cl)c2)cc1O] |
| 5 | 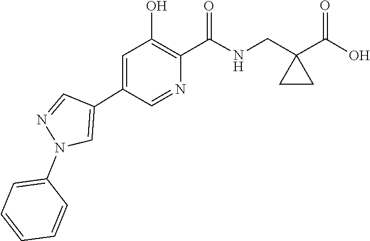 |
| 6 | 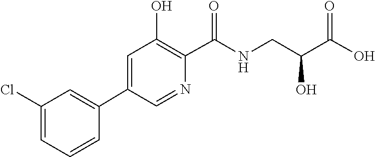 [SMILES: O=C(NC[C@H](O)C(=O)O)c1ncc(-c2cccc(Cl)c2)cc1O] |
| 7 | 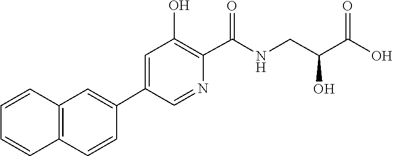 [SMILES: O=C(NC[C@H](O)C(=O)O)c1ncc(-c2ccc3ccccc3c2)cc1O] |
| 8 | 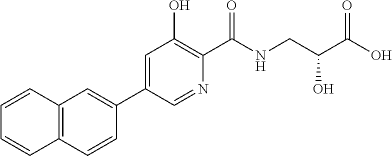 [SMILES: O=C(NC[C@@H](O)C(=O)O)c1ncc(-c2ccc3ccccc3c2)cc1O] |
| 9 | 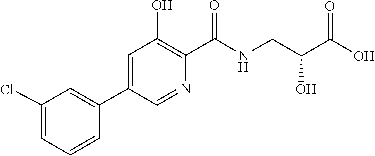 [SMILES: O=C(NC[C@@H](O)C(=O)O)c1ncc(-c2cccc(Cl)c2)cc1O] |
| 10 | 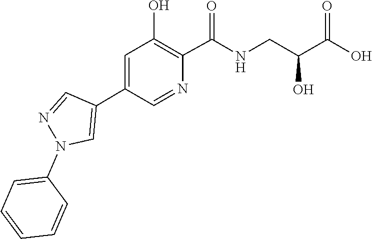 |
| 11 | 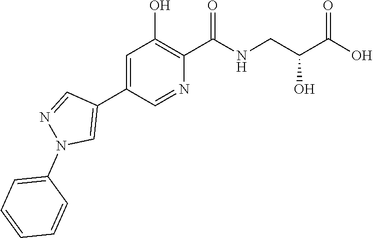 |
| 12 | 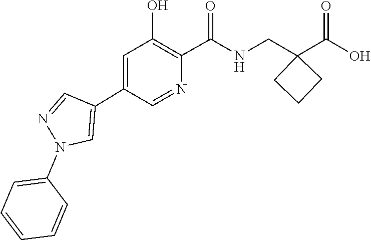 |
| 13 | 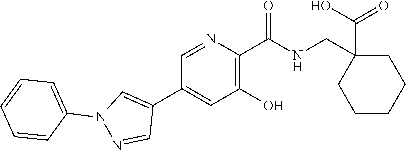 [SMILES: O=C(NCC1(C(=O)O)CCCCC1)c1ncc(-c2cnn(-c3ccccc3)c2)cc1O] |
| 14 | 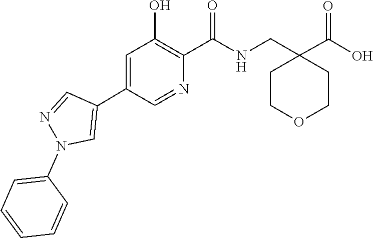 [SMILES: O=C(NCC1(C(=O)O)CCOCC1)c1ncc(-c2cnn(-c3ccccc3)c2)cc1O] |
| 15 | 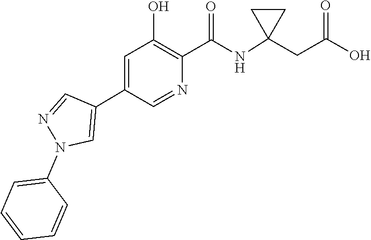 |
| 16 | 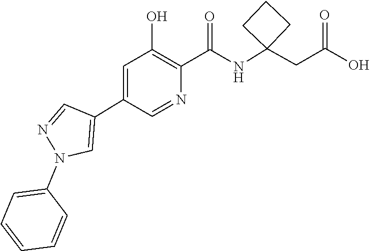 |
| 17 | 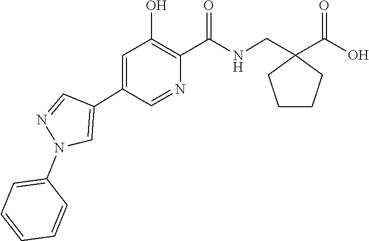 |

34. The compound of any one of claims 1-33, wherein at least one hydrogen atom is replaced with a deuterium atom.

35. A method for treating a disease mediated by PHD1 activity comprising administering to a subject the compound of any one of claims 1-34.

36. The method of claim 35, wherein the disease mediated by PHD1 activity is an ischemic reperfusion injury.

37. The method of claim 35, wherein the ischemic reperfusion injury is selected from stroke, myocardial infarction, and acute kidney injury.

38. The method of claim 35, wherein the disease mediated by PHD1 activity is irritable bowel disease.

39. The method of claim 35, wherein the disease mediated by PHD1 activity is cancer.

40. The method of claim 35, wherein the cancer is colorectal cancer.

41. The method of claim 35, wherein the disease mediated by PHD1 activity is liver disease.

42. The method of claim 35, wherein the disease mediated by PHD1 activity is atherosclerosis.

43. The method of claim 35, wherein the disease mediated by PHD1 activity is cardiovascular disease.
